(19) United States
(12) Patent Application Publication
(10) Pub. No.: US 2014/0328884 A1
(43) Pub. Date: Nov. 6, 2014

(54) CONTROLLED RELEASE VEHICLES HAVING DESIRED VOID VOLUME ARCHITECTURES

(71) Applicant: Celanese EVA Performance Polymers, Inc., Irving, TX (US)

(72) Inventors: Jose Reyes, Irving, TX (US); Kenneth Anderson, Lantana, TX (US); Dale Zevotek, Bethel, OH (US); Nathan Reuter, Hamilton, OH (US); J. Gregory Little, Irving, TX (US); Jeffrey Haley, Norwood, OH (US); Vassilios Galiatsatos, Lebanon, OH (US)

(21) Appl. No.: 14/364,947

(22) PCT Filed: Dec. 17, 2012
(86) PCT No.: PCT/US2012/070058

§ 371 Date: Jun. 12, 2014

Related U.S. Application Data
(60) Provisional application No. 61/576,751, filed on Dec. 16, 2011.
(60) Provisional application No. 61/667,744, filed on Jul. 3, 2012.
(60) Provisional application No. 61/667,680, filed on Jul. 3, 2012.
(60) Provisional application No. 61/667,619, filed on Jul. 3, 2012.

(51) Int. Cl. C08J 9/00; A61K 8/02; A01N 25/10; C05G 3/00; A01N 25/34; A61K 8/81; A61Q 19/00; C08F 218/08; A61K 47/32

(52) U.S. Cl. CPC C08J 9/00; C08F 218/08; A61K 8/0216; A01N 25/10; A61K 47/32; A01N 25/34; A61K 8/8135; A61Q 19/00; C05G 3/0047; C08J 2331/04
USPC 424/401; 521/149; 424/409; 424/422; 71/27

(57) ABSTRACT

Controlled release vehicles may include a polymeric matrix that comprises the polymeric matrix comprising at least one selected from the group consisting of an ethylene copolymer, an ethyl cellulose, a thermoplastic polyurethane, any partially crosslinked polymer thereof, and any combination thereof and has a desired void space architecture.

### BACKGROUND

The present invention relates to controlled release vehicles that may include polymers like ethylene copolymers, ethyl celluloses, and/or thermoplastic polyurethanes that are optionally crosslinked, wherein the controlled release vehicle may have a desired void volume architecture. Further, the present invention relates to the methods, kits, and apparatuses relating to the controlled release vehicles.

In a variety of commercial sectors, vehicles are used to deliver agents to a desired location. As used herein, the term “vehicle” refers to a conveyance for transporting a desired agent. In some cases, the vehicles are designed to release the agent in a controlled manner. As used herein, the term “agent” refers to a payload being delivered, e.g., molecules like iodine contrast agents, compounds like active pharmaceutical agents, and the like. Controlled release can generally be engineered for active controlled release (e.g., by action of another component) or a passive controlled release (e.g., by passage of time).

Active controlled releases often use an external trigger to release the agents from the vehicles. Active controlled release vehicles, such as liposomes and microspheres, can be used to deliver active pharmaceuticals, such as chemotherapeutics, to a desired location in the body, like a tumor. Release of the active pharmaceutical can be triggered by ultrasound, for example, that destabilizes the walls of the liposomes or microspheres, thereby releasing the active pharmaceutical from the liposomes or microspheres. In a pharmaceutical context, active controlled release is believed to provide an effective treatment at the desired location with reduced side effects as the active pharmaceutical is accessible to less of the body. However, active controlled release is often more labor and time intensive, for example, in pharmaceutical delivery where administration, active release, and follow up may be required. Further, active controlled release may have the risk of premature triggering, which may lead to more intense side effects, or trigger failure, which may result in essentially no therapeutic effect.

Passive controlled release often uses void space, degradable polymers, and/or diffusion from and/or through a polymeric matrix to control the release rate of agents from vehicles. Void space is typically formed using a pore forming compound, e.g., surfactants. However, using such methods can provide limited control over the structure of the void space, e.g., morphology and/or interconnectivity. For example, a void space formed with a pore forming compound often has cells (or voids) with a wide diameter distribution and an inconsistent morphology. This can lead to unpredictability in and limited tailoring of release profiles for agents from the controlled release vehicles. Additionally having limited control over the formation of a void space can lead to limited design capabilities in the controlled release of agents. For example, it may be necessary to develop a procedure and a new pore forming compound to design different release profiles for the same agent.

Another method of passive controlled release includes doping degradable polymers with a desired agent. The degradation rate of the polymer is the predominant factor in engineering the release rate of the agent. As the release mechanism depends on the polymer degradation rate, only simple, typically constant, release rates are available with this method. Further, in some cases, the resultant degradation products (e.g., acids) may interact with the agents being delivered, and in some cases, inactivate the agents or produce other undesirable effects.

A third avenue for passive controlled release of agents involves changing the morphology of the polymeric matrix of the vehicle. This general mechanism relies on the agent first solubilizing in the polymeric matrix and then diffusing through the polymeric matrix of the vehicle to the surrounding environment. By increasing the degree of crystallinity of the polymeric matrix, the agents diffuse more slowly through the polymeric matrix, thereby yielding a reduced release rate. However, diffusion through a polymeric matrix may be limited to relatively lower molecular weight agents as higher molecular weight agents either do not diffuse through the polymeric matrix or diffuse too slowly to be effective for a given application. Further, if the agent itself crystallizes in the polymeric matrix, the agent may not be available for diffusion.

Therefore, passive control release vehicles with capabilities, like complex release profiles and controlled release of large molecular weight agents, would be of use to one skilled in the art. Further, methods of producing the vehicles with control would be of use to one skilled in the art.

### SUMMARY OF THE INVENTION

The present invention relates to controlled release vehicles that may include polymers like ethylene copolymers, ethyl celluloses, and/or thermoplastic polyurethanes that are optionally crosslinked, wherein the controlled release vehicle may have a desired void volume architecture. Further, the present invention relates to the methods, kits, and apparatuses relating to the controlled release vehicles.

Some embodiments of the present invention provide a controlled release vehicle comprising: a polymeric matrix having a void space architecture being a substantially interconnected and optionally a polymeric layer disposed on at least a portion of the surface of the polymeric matrix. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a controlled release vehicle comprising: a polymeric matrix having a void space architecture having a bimodal void diameter distribution and optionally a polymeric layer disposed on at least a portion of the surface of the polymeric matrix. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a controlled release vehicle comprising: a polymeric matrix having a void space architecture having an average void diameter of about 500 microns or less and a void diameter distribution having a full width at half max of about 50% or less of the average void diameter and optionally a polymeric layer disposed on at least a portion of the surface of the polymeric matrix. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a controlled release vehicle comprising: a polymeric matrix having a void space architecture having an average void distance of about 250 microns or less and a void distance distribution having a full width at half max of about 75% or less of the average void distance and optionally a polymeric layer disposed on at least a portion of the surface of the polymeric matrix. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a bicomponent controlled release vehicle comprising: a first component and a second component. The first component comprises a polymeric matrix having a void space architecture. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a bicomponent, dual-acting vehicle comprising: a first component, a second component, and optionally a polymeric layer disposed on at least a portion of the surface of the polymeric matrix. The first component comprises a first agent and a polymeric matrix having a first void space architecture. The second component comprises a second agent and a second matrix having a second void space architecture. The first and second polymer matricies independently comprise at least one selected from the group consisting of an ethylene copolymer, an ethyl cellulose, a thermoplastic polyurethane, any partially crosslinked polymer thereof, and any combination thereof.

Some embodiments of the present invention provide a method comprising: providing a polymer melt comprising at least one selected from the group consisting of an ethylene copolymer, an ethyl cellulose, a thermoplastic polyurethane, any partially crosslinked polymer thereof, and any combination thereof; extruding the polymer melt through an extruder; introducing a fluid into the polymer melt while in the extruder; and forming a controlled release vehicle comprising a polymer matrix having a void space architecture.

Some embodiments of the present invention provide a method comprising: providing a controlled release vehicle and administering the controlled release vehicle to a patient. The controlled release vehicle comprises a polymeric matrix having a void space architecture, an agent, and optionally a polymeric layer disposed on at least a portion of the surface of the polymeric matrix such that the controlled release vehicle is for the treatment, prevention, and/or mitigation of a disease or a side effect thereof. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a kit comprising: a set of instructions and a controlled release vehicle. The controlled release vehicle comprises a polymeric matrix having a void space architecture, an agent, and optionally a polymeric layer disposed on at least a portion of the surface of the polymeric matrix such that the controlled release vehicle is for the treatment, prevention, and/or mitigation of a disease or a side effect thereof. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide an in vivo implant comprising: a controlled release vehicle. The controlled release vehicle comprises a polymeric matrix having a void space architecture, an agent, and optionally a polymeric layer disposed on at least a portion of the surface of the polymeric matrix such that the controlled release vehicle is for the treatment, prevention, and/or mitigation of a disease or a side effect thereof. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a personal care product comprising: a controlled release vehicle and at least one selected from the group consisting of a lotion, a cream, a cosmetic, a lipstick, a lip gloss, a deodorant, and any combination thereof. The controlled release vehicle comprises a polymeric matrix having a void space architecture, an agent, and optionally a polymeric layer disposed on at least a portion of the surface of the polymeric matrix. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a container comprising: at least one plastic wall and a controlled release vehicle. The controlled release vehicle comprises a polymeric matrix having a void space architecture, an agent, and optionally a polymeric layer disposed on at least a portion of the surface of the polymeric matrix. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a container comprising: an edible product and a controlled release vehicle. The controlled release vehicle comprises a polymeric matrix having a void space architecture, an agent, and optionally a polymeric layer disposed on at least a portion of the surface of the polymeric matrix. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a fertilizer comprising: a plurality of plant nutrients and a controlled release vehicle. The controlled release vehicle comprises a polymeric matrix having a void space architecture, an agent, and optionally a polymeric layer disposed on at least a portion of the surface of the polymeric matrix. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a kit comprising: a set of instructions and a fertilizer comprising a plurality of plant nutrients and a controlled release vehicle. The controlled release vehicle comprises a polymeric matrix having a void space architecture, an agent, and optionally a polymeric layer disposed on at least a portion of the surface of the polymeric matrix. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a smoking device comprising: a smoking device filter or section thereof that comprises a controlled release vehicle. The controlled release vehicle comprises a polymeric matrix having a void space architecture, an agent, and optionally a polymeric layer disposed on at least a portion of the surface of the polymeric matrix. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide an insect repellent comprising: a controlled release vehicle. The controlled release vehicle comprises a polymeric matrix having a void space architecture, an agent that comprises an insect repellent, and optionally a polymeric layer disposed on at least a portion of the surface of the polymeric matrix. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a controlled release vehicle comprising: a polymeric matrix having a void space architecture having at least one characteristic selected from the group consisting of a bimodal void diameter distribution, an average void diameter of about 500 microns or less, an average void diameter of about 500 microns or less and a void diameter distribution having a full width at half max of about 50% or less of the average void diameter, an average void distance of about 250 microns or less, an average void distance of about 250 microns or less and a void distance distribution having a full width at half max of about 75% or less of the average void distance, an average pore diameter of about 100 microns or less, an average pore diameter of about 100 microns or less and a pore diameter distribution having a full width at half max of about 50% or less of the average pore diameter, a void space volume of about 95% or less, void density of about 1000 voids per cm³ or greater, and any combination thereof. The polymer matrix comprises at least one selected from the group consisting of an ethylene copolymer, an ethyl cellulose, a thermoplastic polyurethane, any partially crosslinked polymer thereof, and any combination thereof.

Some embodiments of the present invention provide a controlled release vehicle comprising: a polymeric matrix having a void space architecture having at least one characteristic selected from the group consisting of open cell, substantially open cell, substantically closed cell, closed cell, any hybrid thereof, and any void space architecture therebetween. The polymer matrix comprises at least one selected from the group consisting of an ethylene copolymer, an ethyl cellulose, a thermoplastic polyurethane, any partially crosslinked polymer thereof, and any combination thereof.

One embodiment of the present invention may provide for a method that comprises: irradiating with an electron beam a plurality of polymer pellets comprising an ethylene vinyl acetate copolymer so as to form a partially crosslinked ethylene vinyl acetate copolymer; melting the partially crosslinked ethylene vinyl acetate copolymer so as to produce a polymer melt; extruding the polymer melt through an extruder; introducing a void forming fluid into the polymer melt while in the extruder; and forming a controlled release vehicle comprising a polymeric matrix having a void space architecture, the polymeric matrix comprising the partially crosslinked ethylene vinyl acetate copolymer.

Another embodiment of the present invention may provide for a method that comprises: extruding a polymer melt through an extruder, the polymer melt comprising ethylene vinyl acetate copolymer; irradiating the polymer melt while in the extruder so as to form a partially crosslinked ethylene vinyl acetate copolymer; introducing a void forming fluid into the polymer melt while in the extruder; and forming a controlled release vehicle comprising a polymeric matrix having a void space architecture, the polymeric matrix comprising the partially crosslinked ethylene vinyl acetate copolymer.

Another embodiment of the present invention may provide for a method that comprises: providing a first polymer melt comprising a first ethylene vinyl acetate copolymer having a first vinyl acetate content; providing a second polymer melt comprising a second ethylene vinyl acetate copolymer having a second vinyl acetate content lower than the first vinyl acetate content; providing a first extruder and a second extruder operably connected such that a second extrudate from the second extruder is disposed on at least a portion of the surface of a first extrudate from the first extruder; extruding the first polymer melt through a first extruder; introducing a void forming fluid into the first polymer melt while in the first extruder; extruding the second polymer melt through a second extruder; forming a controlled release vehicle comprising a polymeric matrix having a void space architecture and a polymeric layer, the polymeric matrix being formed from the first polymer melt in the polymeric layer being formed from the second polymer melt.

Yet another embodiment of the present invention may provide for a method that comprises: providing a first polymer melt; providing a second polymer melt; providing a first extruder and a second extruder operably connected such that a second extrudate from the second extruder is disposed on at least a portion of the surface of a first extrudate from the first extruder; extruding the first polymer melt through a first extruder; introducing a void forming fluid into the first polymer melt while in the first extruder; extruding the second polymer melt through a second extruder; forming a controlled release vehicle comprising a polymeric matrix having a void space architecture and a polymeric layer, the polymeric matrix being formed from the first polymer melt in the polymeric layer being formed from the second polymer melt. The first polymer melt and second polymer melt independently comprise at least one selected from the group consisting of an ethylene copolymer, an ethyl cellulose, a thermoplastic polyurethane, any partially crosslinked polymer thereof, and any combination thereof.

Another embodiment of the present invention may provide for a method that comprises: providing a polymer melt comprising a first ethylene vinyl acetate copolymer having a first vinyl acetate content; extruding the polymer melt through an extruder; introducing a void forming fluid into the polymer melt while in the extruder; forming a polymeric matrix having a void space architecture; and coating at least a portion of the surface of the polymeric matrix so as to form a controlled release vehicle that comprises the polymeric matrix and a polymeric layer disposed on at least a portion of the surface of the polymeric matrix.

Yet another embodiment of the present invention may provide for a method that comprises: providing a polymer melt; extruding the polymer melt through an extruder; introducing a void forming fluid into the polymer melt while in the extruder; forming a polymeric matrix having a void space architecture; and coating at least a portion of the surface of the polymeric matrix so as to form a controlled release vehicle that comprises the polymeric matrix and a polymeric layer disposed on at least a portion of the surface of the polymeric matrix. The polymer melt and polymer layer independently comprise at least one selected from the group consisting of an ethylene copolymer, an ethyl cellulose, a thermoplastic polyurethane, any partially crosslinked polymer thereof, and any combination thereof.

The features and advantages of the present invention will be readily apparent to those skilled in the art upon a reading of the description of the preferred embodiments that follows.

### BRIEF DESCRIPTION OF THE DRAWINGS

The following figures are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

FIGS. 1A-D provide illustrations of at least some void architecture parameters discussed herein.

FIGS. 2A-D provide illustrative cross-sections of nonlimiting examples of void space architectures for controlled release vehicles, or portions thereof, according to at least some embodiments of the present invention.

FIG. 3 provides an illustration of the full-width-at-half-max of a distribution.

FIGS. 4A-B provide illustrative nonlimiting examples of continuous systems for use in conjunction with forming controlled release vehicles, or portions thereof, according to at least some embodiments of the present invention.

FIG. 5 provides an illustrative nonlimiting example of a continuous system for use in conjunction with forming controlled release vehicles, or portions thereof, according to at least some embodiments of the present invention.

FIG. 6 provides an illustrative nonlimiting example of a batch system for use in conjunction with forming controlled release vehicles, or portions thereof, according to at least some embodiments of the present invention.

FIG. 7 provides an illustrative nonlimiting example of a continuous system for use in conjunction with forming controlled release vehicles, or portions thereof, according to at least some embodiments of the present invention having complex macrostructures.

### DETAILED DESCRIPTION

The present invention relates to controlled release vehicles that may include polymers like ethylene copolymers, ethyl celluloses, and/or thermoplastic polyurethanes that are optionally crosslinked, wherein the controlled release vehicle may have a desired void volume architecture. Further, the present invention relates to the methods, kits, and apparatuses relating to the controlled release vehicles.

The present invention provides controlled release vehicles with tailorable capabilities like controlled release of multiple agents, complex release profiles of one or more agents, controlled release of high molecular weight agents, and enhanced capabilities beyond controlled release, like tracking the vehicles and removal of fluid components. These characteristics of the present invention may be useful in various applications including, but not limited to, pharmaceutical release, insecticide release, nutrient release, flavorant and aroma release, preservative release, toxin uptake, and any combination thereof. For example, broadening the capabilities of controlled release to high molecular weight agents (e.g., greater than about 1,000 amu) may be advantageous in the release of biological molecules and chemical catalysts. Other applications may be apparent to those skilled in the art with the benefit of this disclosure.

The components and methods of the present invention may be particularly advantageous in pharmaceutical applications as the need for personalized medicine continues to increase. By way of nonlimiting example, personalized medicine may include preventative treatments based on genetic markers. Using genetic markers may, in some instances, be used to provide more gradation of a disease's progression. With more gradation may come more need for greater control of release rates and, perhaps, complex release profiles. In some instances, the void volume architecture may allow for the use of larger personalized therapeutics, e.g., high molecular weight proteins, antibodies, and potentially stem cells.

The compositions and methods of the present invention provide, in some embodiments, controlled release vehicles having complex release profiles and may be used to control the release of multiple agents. Complex release profiles and controlled release of multiple agents, in a pharmaceutical context, may advantageously provide a mechanism by which complex pharmaceutical therapies may be administered. By way of nonlimiting example, condensing the complex timing of taking multiple medications that mitigate HIV progression to AIDS into perhaps a single daily oral tablet comprising a controlled release vehicle of the present invention may be advantageous. Another example where the controlled release vehicles of the present invention may be particularly useful is in the controlled release of highly addictive pharmaceuticals. By way of nonlimiting example, to reduce the exposure to highly addictive pain medications, a controlled release vehicle of the present invention may be designed to administer an initial bolus of a highly addictive pain medication, e.g., oxycodone, and continuous administration of a less addictive medication to maintain pain relief, e.g., acetaminophen.

The present invention also provides for methods and apparatuses for producing the controlled release vehicles, methods of administering the controlled release vehicles, various kits containing the controlled release vehicles, and articles containing the controlled release vehicles.

The methods of the present invention for producing controlled release vehicles of the present invention may advantageously, in some embodiments, provide for greater control of the architecture of controlled release vehicles, e.g., the void space architecture. The controlled release vehicles of the present invention may also be engineered to have complex macrostructures (discussed further herein) that enable complex release profiles, e.g., of multiple agents. In some embodiments, the engineering control may be aided by changing the melt flow index of the polymers by partially crosslinking the polymers before and/or during the production of the controlled release vehicles. In some embodiments, changing the melt flow index may be done by non-chemical methods, which may be especially advantageous if the agent of the controlled release vehicle is susceptible to reaction with a chemical crosslinker.

The engineering control afforded by at least some embodiments of the present invention may allow for greater control over the release profiles of agents and density, which may affect gastroretentiveness, of the controlled release vehicles. In a pharmaceutical application, density is at least one factor that effects the gastroretentive characteristics of a vehicle, i.e., the length of time a vehicle is in the gastrointestinal tract. In some instances, increased residence time in the gastrointestinal tract provides for improved bioavailability of the agent and/or sustained therapeutic levels over longer time periods, which may in turn, increase therapeutic efficacy and patient compliance.

Other advantages and application of the present invention may be evident to a person having ordinary skill in the art with the benefit of this disclosure.

It should be noted that when “about” is provided at the beginning of a numerical list in this description, “about” modifies each number of the numerical list. It should be noted that in some numerical listings of ranges, some lower limits listed may be greater than some upper limits listed. One skilled in the art will recognize that the selected subset will require the selection of an upper limit in excess of the selected lower limit.

### I. Void Space Architecture of Controlled Release Vehicles

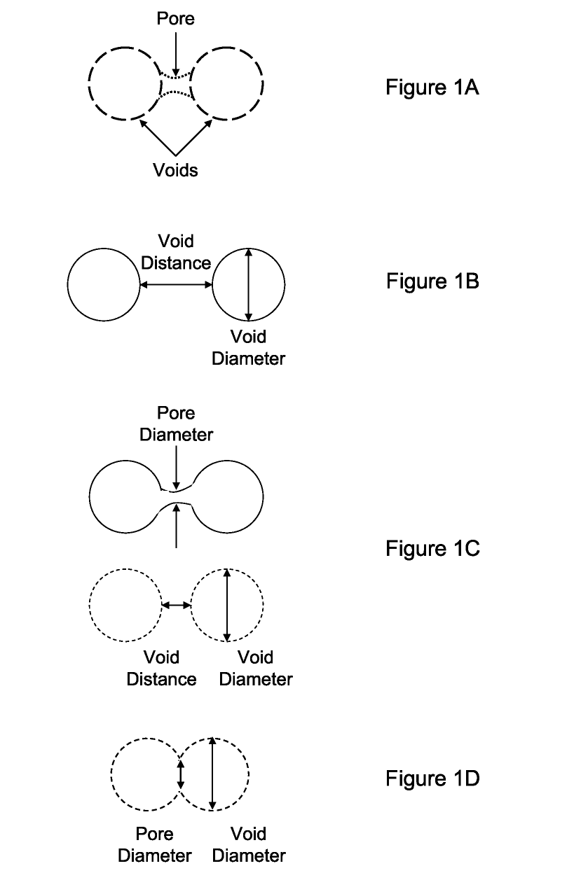

Controlled release vehicles of the present invention may, in some embodiments, include a polymeric matrix having a desired void space architecture. The void space architectures may be defined by parameters including, but not limited to, void diameters, void distances, pore diameters, void space volume, void density, and any combination thereof. FIGS. 1A-D provide illustrations of examples of such parameters. FIG. 1A provides an exemplary illustration of the terms “void” and “pore.” The term “void,” as used herein, refers to a volume not filled with the polymeric matrix within a controlled release vehicle of the present invention. The term “pore,” as used herein, refers to the connection between at least two voids within a controlled release vehicle of the present invention. The term “void diameter,” as used herein, refers to the largest distance between walls of the void, e.g., the diameter in the case of a spherical void, as shown in nonlimiting examples illustrated in FIGS. 1B-D. The term “void distance,” as used herein, refers to the shortest distance between the wall of a void and the wall of a neighboring void, as shown in nonlimiting examples illustrated in FIGS. 1B-C. The term “pore diameter,” as used herein, refers to the shortest distance between the walls of the pore, as shown in nonlimiting examples illustrated in FIGS. 1C-D. It should be noted that two voids connected by a pore may be characterized by a void distance by extrapolating the walls of the voids to a closed void and measuring a distance between the extrapolated walls, as shown in the nonlimiting example illustrated in FIG. 1C. If the extrapolated walls overlap or touch, then the void distance would be considered to be zero, as shown in the nonlimiting example illustrated in FIG. 1D. The term “void space volume,” as used herein, refers to the volume of the void space. The term “void density,” as used herein, refers to the number of voids per unit volume.

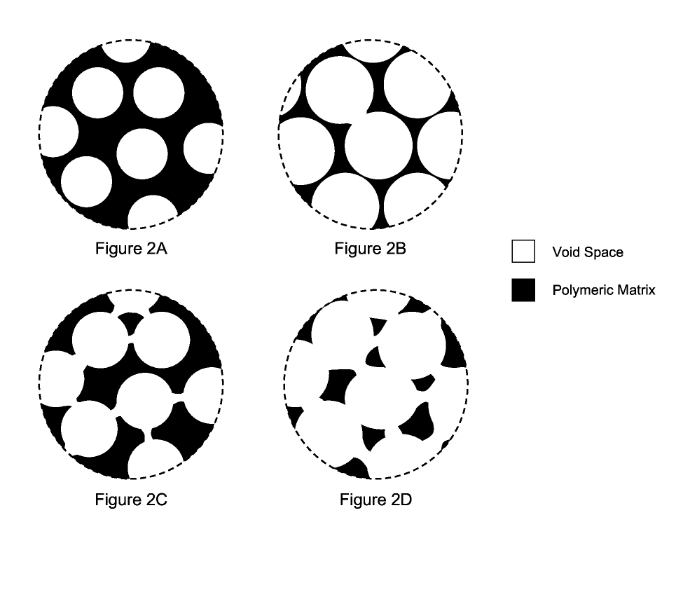

Nonlimiting examples of the void space architectures may include open cell, substantially open cell, substantically closed cell, closed cell, any hybrid thereof, and any void space architecture therebetween. By way of nonlimiting examples of at least some embodiments of the present invention, FIGS. 2A-D provide illustrative cross-sections of void space architectures for controlled release vehicles, or portions thereof, of the present invention. FIG. 2A illustrates a nonlimiting example of a void space architecture for controlled release vehicles of the present invention having discrete voids and may be referred to as a “closed cell” void space architecture, which as used herein refers to 95% or greater of the voids being discrete voids (i.e., not being connected to a neighboring void by a pore). FIG. 2B illustrates a nonlimiting example of a void space architecture for controlled release vehicles of the present invention having substantially discrete voids and may be referred to as a “substantially closed cell” void space architecture, which as used herein refers to about 50% or greater of the voids being discrete voids. FIG. 2C illustrates a nonlimiting example of a void space architecture for controlled release vehicles of the present invention having substantially interconnected voids and may be referred to as a “substantially open cell” void space architecture, which as used herein refers to greater than 50% of the voids being connected to at least one neighboring void by at least one pore. FIG. 2D illustrates a nonlimiting example of a void space architecture for controlled release vehicles of the present invention having interconnected voids and may be referred to as an “open cell” void space architecture, which as used herein refers to about 95% or greater of the voids being connected to at least one neighboring void by at least one pore.

In some embodiments of controlled release vehicles of the present invention, a desired void space architecture of the controlled release vehicles may be characterized by an average void diameter of about 500 microns or less. In some embodiments of controlled release vehicles of the present invention, a desired void space architecture of the controlled release vehicles may be characterized by an average void diameter of about 100 microns or less. In some embodiments of controlled release vehicles of the present invention, a desired void space architecture of the controlled release vehicles may be characterized by an average void diameter of about 10 microns or less. In some embodiments of controlled release vehicles of the present invention, a desired void space architecture of the controlled release vehicles may be characterized by an average void diameter of about 1 micron or less. In some embodiments of controlled release vehicles of the present invention, a desired void space architecture of the controlled release vehicles may be characterized by an average void diameter ranging from a lower limit of about 1 nm, 5 nm, 10 nm, 50 nm, 100 nm, 250 nm, 500 nm, 1 micron, 10 microns, 50 microns, or 100 microns to an upper limit of about 500 microns, 250 microns, 100 microns, 50 microns, 10 microns, 1 micron, or 500 nm, and wherein the average void diameter may range from any lower limit to any upper limit and encompass any subset therebetween.

In some embodiments of the present invention, the controlled release vehicles may have a desired void space architecture that has a bimodal void diameter distribution. In some embodiments of the present invention, a desired void space architecture of the controlled release vehicles may have a bimodal distribution with at least one mode having an average void diameter ranging from a lower limit of about 100 nm, 250 nm, 500 nm, 1 micron, 10 microns, 50 microns, or 100 microns to an upper limit of about 500 microns, 250 microns, 100 microns, 50 microns, 10 microns, 1 micron, or 500 nm, and wherein the average void diameter of the at least one mode may range from any lower limit to any upper limit and encompass any subset therebetween.

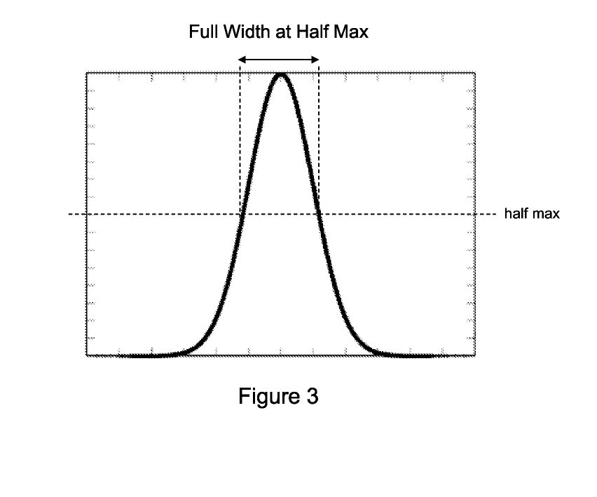

In some embodiments of the present invention, a desired void space architecture of the controlled release vehicles may be characterized by a void diameter polydispersity measured by the full width at half max of the void diameter distribution (or full width at half max of the modes in bimodal distribution embodiments). Full width at half max, as used herein, refers to the width of a distribution at half the maximum intensity of the distribution of some measurement, e.g., average void diameter, where the distribution is the Gaussian curve of the measurement distribution (or multiple Gaussian curves in multi-modal systems). FIG. 3 provides an illustration of the full width at half max of a distribution.

In some embodiments of the present invention, a desired void space architecture of the controlled release vehicles may be characterized by a void diameter distribution having a full width at half max of about 50% or less of the average void diameter, or more preferably about 30% or less of the average void diameter. In some embodiments of the present invention, the full width of half max of the void diameter distribution of the controlled release vehicles may range from a lower limit of about 5%, 10%, or 20% of the average void diameter to an upper limit of about 50%, 40%, 30%, 20%, or 10% of the average void diameter, and wherein the full width at half max of the void diameter distribution may range from any lower limit to any upper limit and encompass any subset therebetween. Further, in some embodiments of the present invention with bimodal void diameter distributions, at least one mode of the diameter distribution may have a full width of half max ranging from a lower limit of about 5%, 10%, or 20% of the average void diameter to an upper limit of about 50%, 40%, 30%, 20%, or 10% of the average void diameter, and wherein the full width at half max of the void diameter distribution may range from any lower limit to any upper limit and encompass any subset therebetween.

In some embodiments of the present invention, a desired void space architecture of the controlled release vehicles may be characterized by an average void distance of about 250 microns or less. In some embodiments of the present invention, a desired void space architecture of the controlled release vehicles may be characterized by an average void distance of about 100 microns or less. In some embodiments of the present invention, a desired void space architecture of the controlled release vehicles may be characterized by an average void distance of about 10 microns or less. In some embodiments of the present invention, a desired void space architecture of the controlled release vehicles may be characterized by an average void distance of about 1 micron or less. In some embodiments of the present invention, a desired void space architecture of the controlled release vehicles may be characterized by an average void distance of about 100 nm or less. In some embodiments of the present invention, a desired void space architecture of the controlled release vehicles may be characterized by an average void distance ranging from a lower limit of about zero (i.e., touching or overlapping voids), 25 nm, 100 nm, 250 nm, 500 nm, 1 micron, 10 microns, or 50 microns to an upper limit of about 250 microns, 100 microns, 50 microns, 10 microns, 1 micron, or 500 nm, and wherein the average void distance may range from any lower limit to any upper limit and encompass any subset therebetween.

In some embodiments of the present invention, a desired void space architecture of the controlled release vehicles may be characterized by a void distance polydispersity measured by the full width at half max of the void distance distribution. In some embodiments of the present invention, a desired void space architecture of the controlled release vehicles may be a void distance distribution having a full width at half max of about 75% or less of the average void distance, about 50% or less of the average void distance, or more preferably about 30% or less of the average void distance. In some embodiments of the present invention, the full width of half max of the void distance distribution of the controlled release vehicles may range from a lower limit of about 5%, 10%, or 20% of the average void distance to an upper limit of about 75%, 50%, 40%, 30%, 20%, or 10% of the average void distance, and wherein the full width at half max of the void distance distribution may range from any lower limit to any upper limit and encompass any subset therebetween.

In some embodiments of the present invention, a desired void space architecture of the controlled release vehicles may be characterized by an average pore diameter of about 100 microns or less. In some embodiments of the present invention, a desired void space architecture of the controlled release vehicles may be characterized by an average pore diameter of about 10 microns or less. In some embodiments of the present invention, a desired void space architecture of the controlled release vehicles may be characterized by an average pore diameter of about 1 micron or less. In some embodiments of the present invention, a desired void space architecture of the controlled release vehicles may be characterized by an average pore diameter of about 100 nm or less. In some embodiments of the present invention, a desired void space architecture of the controlled release vehicles may be characterized by an average pore diameter ranging from a lower limit of 25 nm, 100 nm, 250 nm, 500 nm, 1 micron, or 10 microns to an upper limit of about 100 microns, 50 microns, 10 microns, 1 micron, 500 nm, or 250 nm, and wherein the average pore diameter may range from any lower limit to any upper limit and encompass any subset therebetween.

In some embodiments of the present invention, a desired void space architecture of the controlled release vehicles may be characterized by a pore diameter polydispersity measured by the full width at half max of the pore diameter distribution. In some embodiments of the present invention, a desired void space architecture of the controlled release vehicles may be characterized by a pore diameter distribution having a full width at half max of about 50% or less of the average pore diameter, about 30% or less of the average pore diameter, or more preferably about 20% or less of the average pore diameter. In some embodiments of the present invention, the full width of half max of the pore diameter distribution of the controlled release vehicles may range from a lower limit of about 5%, 10%, or 20% of the average pore diameter to an upper limit of about 50%, 40%, 30%, 20%, or 10% of the average pore diameter, and wherein the full width at half max of the pore diameter distribution may range from any lower limit to any upper limit and encompass any subset therebetween.

In some embodiments of the present invention, a desired void space architecture of the controlled release vehicles may be characterized by a void space volume of about 95% or less, about 75% or less, or 50% or less. In some embodiments of the present invention, a desired void space architecture of the controlled release vehicles may be characterized by a void space volume ranging from a lower limit of about 5%, 10%, 25%, 50%, or 75% to an upper limit of about 95%, 90%, 80%, 75%, or 50%, and wherein the void space volume may range from any lower limit to any upper limit and encompass any subset therebetween. It should be noted that void space volume may be converted to other units, for example, 90% void volume space may equate to 0.9 cc/cc void volume space.

In some embodiments of the present invention, a desired void space architecture of the controlled release vehicles may be characterized by a void density of about 1 void per cm³ or greater, 10 voids per cm³ or greater, 100 voids per cm³ or greater, 1000 voids per cm³ or greater, 10,000 voids per cm³ or greater, 100,000 voids per cm³ or greater, 1,000,000 voids per cm³ or greater, or 10 million voids per cm³. In some embodiments of the present invention, a desired void space architecture of the controlled release vehicles may be characterized by a void density ranging from a lower limit of about 1 void per cm³, 10 voids per cm³, 25 voids per cm³, 50 voids per cm³, 100 voids per cm³, 1000 voids per cm³, 10,000 voids per cm³, 100,000 voids per cm³, 1,000,000 voids per cm³ to an upper limit of about 125 trillion voids per cm³, about 1 trillion voids per cm³, about 100 billion voids per cm³, about 1 billion voids per cm³, about 100,000,000 voids per cm³, or about 1,000,000 voids per cm³, and wherein the void density may range from any lower limit to any upper limit and encompass any subset therebetween. One skilled in the art, with the benefit of this disclosure, should understand that the void density will depend on, inter alia, the void diameter and that smaller void diameters allow for higher void densities.

Controlled release vehicles of the present invention may, in some embodiments, include a polymeric matrix having a void space architecture. The void space architectures may optionally be characterized by at least one of the following bimodal void diameter distributions, average void diameter (optionally including polydispersity of the average void diameter), average void distance (optionally including polydispersity of the average void distance), average pore diameter (optionally including polydispersity of the average pore diameter), void space volume, void density, a description of the void space architecture (e.g., open cell, substantially open cell, substantically closed cell, closed cell, any hybrid thereof, and any void space architecture therebetween), and any combination thereof (including combinations of three or more characteristics).

### II. Controlled Release Vehicles Compositions

In some embodiments of the present invention, the polymeric matrix of the controlled release vehicles of the present invention may comprise ethylene copolymers, ethyl celluloses, and/or thermoplastic polyurethanes. In some embodiments, the polymeric matrix of the controlled release vehicles of the present invention may comprise partially crosslinked polymers (e.g., partially crosslinked ethylene copolymers, partially crosslinked ethyl cellulose, and/or partially crosslinked thermoplastic polyurethane, alone or in any combination). As used herein, the term “partially crosslinked” refers to a polymer having at least some crosslinks, such that the degree of crosslinking is below the Flory gel point of the polymer and the polymer being capable of undergoing viscous flow. In some embodiments, the polymeric matrix of the controlled release vehicles of the present invention may comprise both partially crosslinked and non-partially crosslinked polymers (e.g., ethylene copolymers, ethyl celluloses, and/or thermoplastic polyurethanes). For simplicity, when describing various embodiments of the present invention, ethylene copolymers, ethyl celluloses, and thermoplastic polyurethanes encompass the partially crosslinked versions thereof.

In some embodiments, partially crosslinked polymers of a polymeric matrix described herein may be at least substantially free of chemical crosslinkers. As used herein, the term “substantially free of chemical crosslinkers” refers to a polymer (crosslinked, partially crosslinked, or otherwise) comprising a chemical crosslinker in an amount of about 0.01% or less by weight of the polymer. It is believed that, in some embodiments, a polymeric matrix comprising partially crosslinked polymers that is substantially free of chemical crosslinkers may advantageously minimize degradation and/or inactivation of an agent (described further herein) as a result of reaction with a chemical crosslinker.

Examples of ethylene copolymers may include, but are not limited to, polymers that comprise ethylene monomers and at least one monomer of vinyl acetate, methyl acrylate, ethyl acrylate, n-butyl acrylate, ethyl methacrylate, acrylic acid, methacrylic acid, propylene, 1-butene, 1-pentene, 1-hexene, 1-heptene, 1-octene, 4-methyl-1-pentene, any derivative thereof, and any combination thereof.

In some embodiments, the polymeric matrix of the controlled release vehicles of the present invention may comprise ethylene vinyl acetate copolymers having a vinyl acetate content ranging from a lower limit of greater than 0% or about 9%_{,} 18%, 28%, or 33% to an upper limit of about 42%, 40%, 33%, or 28%, and wherein the vinyl acetate content of the copolymer may range from any lower limit to any upper limit and encompass any subset therebetween.

In some embodiments, the polymeric matrix of the controlled release vehicles of the present invention may comprise ethylene copolymers, ethyl celluloses, and/or thermoplastic polyurethanes and additional thermoplastic polymers. The additional thermoplastic polymers may, in some embodiments, be included as at least a portion of copolymers (including copolymers of more than two polymers, e.g., terpolymers), blend polymers, graft polymers, branched polymers, star polymers, and the like, or any hybrid thereof.

Suitable thermoplastic polymers for use in conjunction with the present invention may include, but are not limited to, polyethylene, polypropylene, acrylic acid polymers, polytetrafluoroethylene (PTFE), ethylene vinyl acetate copolymer derivatives, polyesters, polybutadiene, polyisoprene, poly(methacrylate), poly(methyl methacrylate), styrene-butadiene-styrene block copolymers, poly(hydroxyethylmethacrylate) (pHEMA), poly(vinyl chloride), poly(vinyl acetate), polyethers, polyacrylonitriles, polyethylene glycols, polymethylpentene, polybutadiene, polyhydroxy alkanoates, poly(lactic acid), poly(glycolic acid), acrylic acid-based polymers, methacrylic acid based polymers, cellulosic polymers, polyanhydrides, polyorthoesters, cross-linked poly(vinyl alcohol), neoprene rubber, butyl rubber, alkylcelluloses (e.g., calcium carboxymethyl cellulose, certain substituted cellulose polymers, hydroxypropyl methylcellulose phthalate, hydroxypropyl methylcellulose acetate succinate, cellulose acetate butyrate, cellulose acetate phthalate, cellulose acetate trimaleate), polyvinyl acetate phthalate, polyvinyl acetate, polyester, shellac, zein, polyethylene oxide (PEO), ethylene oxide-propylene oxide copolymers (include block copolymers like PLURONICS® (polyethylene oxide-polypropylene oxide-polyethylene oxide triblock polymers, available from BASF)), polyethylene-polypropylene glycol (e.g., poloxamer), carbomer, polycarbophil, chitosan, polyvinyl pyrrolidone (PVP), poly(vinyl alcohol) (PVA), hydroxyalkyl celluloses (e.g., hydroxypropyl cellulose (HPC), hydroxyethyl cellulose (HEC), hydroxymethyl cellulose, and hydroxypropyl methylcellulose (HPMC)), carboxymethyl cellulose, sodium carboxymethyl cellulose, methylcellulose, hydroxyethyl methylcellulose, hydroxypropyl methylcellulose, polyacrylates, polyacrylamides, polymethacrylamides, polyphosphazines, polyoxazolidines, polyhydroxyalkylcarboxylic acids, alginic acids (e.g., carrageenate alginates, ammonium alginate, and sodium alginate), starch and starch derivatives, polysaccharides, carboxypolymethylene, polyethylene glycol (PEG), natural gums (e.g., gum guar, gum acacia, gum tragacanth, karaya gum, and gum xanthan), povidone, gelatin, and the like, any derivative thereof, any copolymer thereof, any blend polymer thereof, and any combination thereof. In some preferred embodiments, suitable thermoplastic polymers for use in conjunction with the present invention may include, but are not limited to, polyethylene, polypropylene, poly(hydroxyethylmethacrylate) (pHEMA), polyethers, polyethylene glycols, polyhydroxy alkanoates, poly(lactic acid), poly(glycolic acid), polyethylene oxide (PEO), polyvinyl pyrrolidone (PVP), poly(vinyl alcohol) (PVA), hydroxyalkyl celluloses (e.g., hydroxypropyl cellulose (HPC), hydroxyethyl cellulose (HEC), hydroxymethyl cellulose, and hydroxypropyl methylcellulose (HPMC)), polyethylene glycol (PEG), any derivative thereof, any copolymer thereof, any blend polymer thereof, and any combination thereof. Suitable thermoplastic polymers may include, but are not limited to, polyvinyl caprolactam-polyvinyl acetate-PEG graft copolymers like SOLUPLUS® (PEG 6000/vinylcaprolactam/vinyl acetate 13/57/30, available from BASF). As used herein, the term “derivative” refers to any compound that is made from one of the listed compounds, for example, by replacing one atom in the base compound with another atom or group of atoms.

In some embodiments of the present invention, the thermoplastic polymers may be degradable. As used herein, the terms “degrading,” “degradation,” and “degradable” refer to both the relatively extreme cases of degradation that the degradable material may undergo (i.e., bulk erosion and surface erosion) and any stage of degradation in between these two. Suitable degradable thermoplastic polymers for use in conjunction with the present invention may include, but are not limited to, aliphatic polyesters, poly(lactic acid), poly(glycolic acid), poly(lactic-co-glycolic acid), poly(butylene succinate), poly(caprolactone), polyanhydrides, poly(vinyl alcohol), starches, cellulosics, chitans, chitosans, cellulose esters, cellulose acetate, nitrocellulose, and the like, any derivative thereof, and any combination thereof. In some preferred embodiments, suitable degradable thermoplastic polymers for use in conjunction with the present invention may include, but are not limited to, methyl cellulose, poly(lactic acid), poly(glycolic acid), poly(lactic-co-glycolic acid), poly(vinyl alcohol), any derivative thereof, and any combination thereof.

In some embodiments, the polymeric matrix of the controlled release vehicles of the present invention may comprise ethylene copolymers, ethyl celluloses, and/or thermoplastic polyurethanes and a plasticizer. Suitable plasticizers for use in conjunction with the present invention may include, but are not limited to, triacetin, triclosan, citrate-based esters, phthalates, teraphthalates, vegetable oils, and the like, and any combination thereof.

In some embodiments, the controlled release vehicles of the present invention may comprise at least one agent and a polymeric matrix having a void space architecture.

Suitable agents for use in conjunction with the present invention may include, but are not limited to, active agents, removal agents, tracking agents, cellular agents, any hybrid thereof, and any combination thereof. As used herein, the term “active agent” refers to a compound, molecule, particulate, or “pro”-version thereof that actively participates in a biological or chemical pathway. As used herein, the modifier “pro” refers to an article (e.g., compound, molecule, or particulate) that becomes an active agent after a known chemical reaction, whether biologically induced or otherwise. As used herein, the term “removal agent” refers to a compound, molecule, or particulate that is capable of reducing the concentration of a constituent (e.g., another compound, molecule, or particulate) from a fluid, e.g., a chelating agent that removes heavy metal ions. As used herein, the term “tracking agent” refers to a compound, molecule, or particulate that is capable of being tracked, e.g., an x-ray contrast agent like iodine or a nanoparticle that interacts with radio-frequency waves. As used herein, the term “cellular agent” refers to cells and cell-like structures.

Suitable agents for use in conjunction with the present invention may include, but are not limited to, cells, compounds, molecules, particulates, and/or pro-versions thereof that are capable of interacting with biological pathways, biochemical pathways, sensory organs, desired chemical reactions, decomposition reactions, electromagnetic radiation, and any combination thereof. Nonlimiting examples of agents suitable for use in conjunction with the present invention may include, but are not limited to, active pharmaceuticals (e.g., hydrophilic active pharmaceutical, hydrophobic active pharmaceutical, amphoteric active pharmaceutical, pain relievers, antibiotics, steroids, and antioxidants), prodrugs of active pharmaceuticals, active biologicals (e.g., hormones, DNAs, RNAs, siRNAs, peptides, enzymes, nucleotides, oligionucleotides, antibodies, and monoclonal antibodies), antibiotics, antifungals, antitoxins, antigens, therapeutics (e.g., chemotherapeutics, radiation-poisoning therapeutics, radioisotopes), preventive therapeutics (e.g., antioxidants, radiation mitigation agents, and vaccines), nutritional supplements (e.g., vitamins, nutraceuticals, metabolism enhancing agents, and antioxidants), imaging agents (e.g., magnetic resonance imaging contrast agents, x-ray imaging contrast agents, and radioisotopes), fluid stabilizers (e.g., blood-clotting factors and emulsion stabilizers), food agents (e.g., preservatives, fragrances, and aromas), flavorants, olfactory agents (e.g., fragrances and aromas), plant agents (e.g., pesticide and fertilizer), chemical-reaction agents (e.g., chemical crosslinkers and catalysts), insect repellents, cells (e.g., endothelial cells, hepatic cells, myocytes, progenitor cells, stem cells, and parthenogenetic stem cells), and any combination thereof. Additional nonlimiting examples of specific agents are detailed further herein.

It should be noted that some active agents, removal agents, and tracking agents may overlap. By way of nonlimiting example, some chelating agents may actively participate in a biological pathway by making unavailable an ion for reaction, thereby making the chelating agents both active agents and removal agents.

In some embodiments, the controlled release vehicles of the present invention may comprise additional ingredients and a polymeric matrix having a void space architecture. Suitable additional ingredients may include, but are not limited to, bar-code additives, light-emitting agents, colorimetric agents, glidants, anti-adherents, anti-static agents, gums, sweeteners, preservatives, stabilizers, adhesives, pigments, sorbents, nanoparticles, microparticles, lubricants, disintegrants, excipients, powder flow aids, nucleating agents, pore forming compounds, swellable polymers, effervescent materials, physical blowing compounds, bioadhesives, gastroretentive compounds, and any combination thereof. It should be noted that some additional ingredients may fall within more than one category.

As used herein, the term “bar-code additive” refers to an innocuous additive with a unique signature that identifies the controlled release vehicle. Identification may be advantageous for identifying counterfeits, tracking batches of controlled release vehicles, and labeling and extracting batches of controlled release vehicles from a continuous process. Suitable bar-code additives may have, but are not limited to, at least one component comprising a fluorophore, a nanoparticle (e.g., noble metal nanoparticles having a diameter of about 0.5 nm to about 500 nm, core-shell nanoparticles with at least the shell being nano-dimensional, magnetic nanoparticles, quantum dots, carbon nanoparticles, and the like), a radioisotope, and the like, and any combination thereof. Bar-code additives may, in some embodiments, derive their unique signature from several components in a unique concentration relationship. By way of nonlimiting example, a bar-code additive may have 3 nm gold particles, 10 nm gold particles, and 25 nm gold particles with relative concentrations of 1:5:2, thereby enabling the spectroscopic signature of the nanoparticles in that concentration relationship to identify the manufacturer of the controlled release vehicle. By way of another nonlimiting example, a bar-code additive may be a fluorophore encoded via photobleaching, which may be immobilized on a substrate like a glass fiber.

Lubricants suitable for use in conjunction with the present invention may include, but are not limited to, magnesium stearate, and the like, derivatives thereof, and any combination thereof.

Disintegrants suitable for use in conjunction with the present invention may include, but are not limited to, crospovidone, sodium starch glycolate, crosscarmellose sodium, and the like, derivatives thereof, and any combination thereof.

Excipients suitable for use in conjunction with the present invention may include, but are not limited to, microcrystalline cellulose, lactose, mannitol, silica, dicalcium phosphate, starch, maltodextrins, sorbitol, glucitol, xylitol, and the like, derivatives thereof, and any combination thereof.

Powder flow aids may be useful, in some embodiments, for inclusion during the production of the controlled release vehicles of the present invention (described further herein) where at least one precursor (e.g., polymer pellets or agents) are in powder form and processing homogeneity may benefit from the powder flow aid. Powder flow aids suitable for use in conjunction with the present invention may include, but are not limited to, fumed silica, precipitated silica, nano-sized silica, calcium carbonate, precipitated calcium carbonate, nano-sized calcium carbonate, and any combination thereof.

Nucleating agents may, in some embodiments, be useful as, inter alia, providing substantially homogeneously distributed nucleation sites for the formation of voids during the production of controlled release vehicles of the present invention (described further herein). Nucleating agents suitable for use in conjunction with the present invention may include, but are not limited to, fumed silica, precipitated silica, nano-sized silica, nanoclays, and any combination thereof.

Pore forming compounds suitable for use in conjunction with the present invention may include, but are not limited to, at least partically water soluble or degradable polymers like polyethylene glycol, polylactic acid, and the like. In some embodiments, pore forming compounds may be excluded from the controlled release vehicles of the present invention including methods related thereto.

Swellable polymers suitable for use in conjunction with the present invention may include, but are not limited to, hydrogels, hydroxypropyl methylcellulose, carboxy methylcellulose, poly(hydroxyethylmethacrylate), alginic acid, hyaluranic acid, polysaccharides, chitosans, croscarmellose, crospovidone, and the like, and any combination thereof.

Effervescent materials (sometimes referred to as chemical blowing agents) suitable for use in conjunction with the present invention may include, but are not limited to, a carbonate or a bicarbonate like sodium bicarbonate, calcium bicarbonate, potassium bicarbonate, sodium carbonate, calcium carbonate, potassium carbonate, sodium glycine carbonate, azo-compounds, and the like, and any combination thereof.

Physical blowing compounds suitable for use in conjunction with the present invention may include, but are not limited to, n-butane, isobutane, carbon dioxide, nitrogen, and the like, and any combination thereof.

Bioadhesives may advantageously provide for temporary adhesion of a controlled release vehicle to biological tissue. Bioadhesives suitable for use in conjunction with the present invention may include, but are not limited to, cellulose, cellulose derivatives, hydroxyethylcellulose, sodium carboxymethylcellulose, partially crosslinked polyacrylic acid, carboxy vinyl polymers, lectin, alginates, tragacanth gum, carbomers and cornstarch (e.g., PROLOC®, a mix of high molecular weight crosslinked polyacrylic acid and amylopectin, available from Henkel), thiolated polycarbophil, fibrin glud (e.g., a combination of fibrinogen and thrombin), and the like, and any combination thereof.

As used herein, the term “gastroretentive compounds” refer to chemicals that delay gastric emptying. Gastroretentive compounds suitable for use in conjunction with the present invention may include, but are not limited to, narcotic pain relievers, anticholinergic medications, anti-diarrheal compounds, carbohydrate-digestion delay compounds, acarbose, octreotide, and the like, and any combination thereof. It should be noted that some gastroretentive compounds may have serious side effects, and in some embodiments, should be utilized in very low concentrations.

In some embodiments, additional ingredients may be included in a controlled release vehicle of the present invention in an amount ranging from a lower limit of about 0.01%, 0.1%_{,} 1%_{,} 5%, 10%, or 25% by weight of the controlled release vehicle to an upper limit of about 70%, 65%, 55%, or 40% by weight of the controlled release vehicle, and wherein the amount of additional ingredients may range from any lower limit to any upper limit and encompass any subset therebetween.

In some embodiments, the controlled release vehicles of the present invention may comprise a surface coating and a polymeric matrix having a void space architecture. It should be noted that the term “coating” does not imply 100% surface coverage. In some embodiments, the surface coating may be a polymeric layer disposed on at least a portion of the surface of the polymeric matrix having a void space architecture.

Polymers suitable for use in conjunction with surface layers on at least a portion of the surface of a polymeric matrix of a controlled release vehicle of the present invention may include, but are not limited to, ethylene vinyl acetate copolymers, ethyl celluloses, thermoplastic polyurethanes, additional thermoplastic polymers (including those listed above), food-derived polymers, sugars, starches, and the like, any derivative thereof, any copolymer thereof, any blend polymer thereof, and any combination thereof. In some embodiments, a surface layer may comprise a degradable polymer, e.g., those listed above. In some embodiments, a surface layer may comprise a polymeric matrix having or not having a void space architecture described herein.

In some embodiments, a surface layer (e.g., a polymeric layer) may be involved with at least one of: controlling the release profile of an agent, providing burst release in the release profile of an agent, delaying release of an agent, providing protection to the controlled release vehicle, and any combination thereof.

A surface coating (e.g., a polymeric layer) may, in some embodiments, be involved with the release profile of an agent. For example, a patch comprising a controlled release vehicle of the present invention may comprise a first layer comprising a polymeric matrix having a void space architecture and a second layer optionally having a void space architecture, such that an agent can brew released from the first layer faster than the second layer. Accordingly, in at least this example, the first layer may act as a reservoir, while the second layer is involved with the release profile of the agent. In some embodiments, a controlled release vehicle of the present invention may comprise a polymeric matrix that comprises a first ethylene vinyl acetate copolymer and a polymeric layer that comprises a second ethylene vinyl acetate copolymer, wherein the percent vinyl acetate in the second ethylene vinyl acetate copolymer is less than the percent vinyl acetate in the first ethylene vinyl acetate copolymer. By way of nonlimiting example, a vaginal ring for the release agents that mitigate the symptoms of a sexually transmitted disease may comprise (1) an inner core that comprises a first polymeric matrix having a first void space architecture and (2) a surface coating (e.g., polymeric layer) disposed about the inner core, wherein the surface coating comprises a second polymeric matrix having a second void space architecture. Further, in some embodiments of this example, the second polymeric matrix may be designed so as to control the release rate of the agents from the vaginal ring, and the first polymeric matrix may be designed so as to maximize capacity for the agents, thereby increasing the length of time the vaginal ring may be utilized. Design parameters for each of the inner core and surface coating that may provide for such a vaginal ring may include, but are not limited to, respective void space architectures (e.g., the second void space architecture being substantially closed cell and the first void space architecture being substantially open cell), the respective polymeric matricies (e.g., varying the vinyl acetate content as described above), and the like, and any combination thereof.

A surface coating (e.g., a polymeric layer) may, in some embodiments, advantageously provide burst release capabilities to controlled release vehicles of the present invention. By way of nonlimiting example, an oral controlled release vehicle may comprise (1) a core that comprises a first polymeric matrix having a void space architecture and an agent for treatment of acid reflux disease (e.g., esomeprazole) and (2) a polymeric layer disposed about the core, the polymeric layer comprising a degradable polymer and an antacid (e.g., calcium carbonate), such that the degradable polymer degrades in stomach acid to provide a burst release of the antacid. Such an oral controlled release vehicle may advantageously immediately treat the symptoms of heartburn while treating a cause of acid reflux for, potentially, long-term health benefits.

A surface coating (e.g., a polymeric layer) may, in some embodiments, advantageously delay onset of the controlled release and/or uptake capabilities of the controlled release vehicles of the present invention. For example, in a pharmaceutical application, the delay may allow for the controlled release vehicle to be taken orally and delay release of an active agent until in a desired area in a patient, e.g., the gastrointestinal tract of a patient. For example in fertilizer applications, the coating may provide for shipping and placement of the controlled release vehicles before releasing an active agent into the soil.

Polymeric layers disposed on at least a portion of the surface of the polymeric matrix may have a thickness ranging from a lower limit of about 10 microns, 20 microns, or 30 microns to an upper limit of about 100 microns, 90 microns, or 75 microns, and wherein polymeric layer thickness may range from any lower limit to any upper limit and encompass any subset therebetween.

In some embodiments, at least a portion of the surface of a polymeric matrix of a controlled release vehicle of the present invention may have more than one layer. By way of nonlimiting example, the surface of the polymeric matrix of a controlled release vehicle of the present invention may have disposed thereon a first layer with the function of assisting in the controlled release of an agent from the controlled release vehicle and a second layer capable of degrading (e.g., a lactic acid containing polymer) that is disposed on the first layer with the function of mitigating an upset stomach.

In some embodiments, a surface coating (e.g., a polymer layer) of a controlled release vehicle of the present invention may comprise at least one agent (e.g., active agents, removal agents, tracking agents, and any combination thereof). In some embodiments, a surface coating (e.g., a polymer layer) of a controlled release vehicle of the present invention may further comprise at least bar-code additives, light-emitting agents, colorimetric agents, glidants, anti-adherents, anti-static agents, flavorants, gums, sweeteners, preservatives, stabilizers, adhesives, pigments, sorbents, nanoparticles, microparticles, lubricants, disintegrants, excipients, powder flow aids, nucleating agents, pore forming compounds, swellable polymers, effervescent materials, physical blowing compounds, bioadhesives, gastroretentive compounds, and any combination thereof. By way of nonlimiting example, a surface coating of a controlled release vehicle described herein may, in some embodiments, comprise antioxidants, which may provide for long-term storage of the controlled release vehicle by mitigating oxidative damage to agents in the controlled release vehicle.

In some embodiments, controlled release vehicles of the present invention may have a complex macrostructure. As used herein, the term “macrostructure” refers to the overall organization of the controlled release vehicle. In some embodiments, the controlled release vehicles of the present invention may have a multi-component (e.g., bicomponent) macrostructure. Examples of possible multi-component macrostructures of the controlled release vehicles of the present invention may include, but are not limited to, side-by-side, sheath-core (e.g., in the form of a layer disposed on at least a portion of the surface of a controlled release vehicle), concentric core-sheath, eccentric core-sheath, concentric spheres, eccentric spheres, trapezoidal, segmented-pie, islands-in-the-sea, three islands-in-the-sea, tipped, segmented-ribbon, or any hybrid thereof.

In some embodiments, controlled release vehicles of the present invention may have at least one component of a multi-component macrostructure comprising a polymeric matrix and a void space having a desired architecture according to any of the embodiments described herein. In some embodiments, a controlled release vehicle of the present invention may have at least two components of a multi-component macrostructure comprising a polymeric matrix and a void space having a desired architecture according to any of the embodiments described herein where each component differs in at least the polymeric matrix or the void space architecture. By way of nonlimiting example, a controlled release vehicle of the present invention may have a first component and a second component each with polymeric matrices and void space architectures according to any embodiment described herein with the polymer matrices differing, the void space architectures differing, or both the polymer matrices and the void space architectures differing. By way of another nonlimiting example, a controlled release vehicle of the present invention may have a core component and a surface layer component disposed on at least a portion of the surface of the core component, such that each have different polymer matrices (e.g., ethylene vinyl acetate copolymers with differing percent vinyl acetate content), different agents disposed therein (e.g., a small molecule active agent in the surface layer and a larger peptide in the core), or a combination thereof.

In some embodiments, controlled release vehicles of the present invention may have at least one component of a multi-component macrostructure comprising a polymeric matrix and a void space having a desired architecture according to any of the embodiments described herein and at least one component of the multi-component macrostructure comprising an additional thermoplastic polymer (and/or a degradable polymer) described above. In some embodiments, the at least one component comprising the additional thermoplastic polymer (and/or the degradable polymer) described above may optionally include plasticizers, additional ingredients, and any combination thereof. By way of nonlimiting example, a core-sheath controlled release vehicle may have a sheath according to an embodiment described herein (i.e., having a polymeric matrix and a void space having a desired architecture) and a core of a degradable polymer (e.g., poly(lactic acid)).

In some embodiments, controlled release vehicles of the present invention may have a density ranging from a lower limit of about 0.1 g/cm³, 0.25 g/cm³, 0.5 g/cm³, 0.6 g/cm³, or 0.7 g/cm³ to an upper limit of about 0.97 g/cm³, 0.95 g/cm³, or 0.9 g/cm³, and wherein the density may range from any lower limit to any upper limit and encompass any subset therebetween. It should be understood by one of ordinary skill in the art that the density of a controlled release vehicle may be engineered with changes to, inter alia, the void space architecture, the composition, and the like.

In some embodiments, controlled release vehicles (or portions thereof) of the present invention may comprise a polymeric matrix having a void space architecture in any combination of polymeric matrices and void space architecture of embodiments described herein. In some embodiments, the controlled release vehicles of the present invention may optionally further comprise (alone or in any combination) additional thermoplastic polymers, degradable thermoplastic polymers, plasticizers, agents, additional ingredients, and surface coatings (e.g., a polymeric layer).

### III. Forming Controlled Release Vehicles

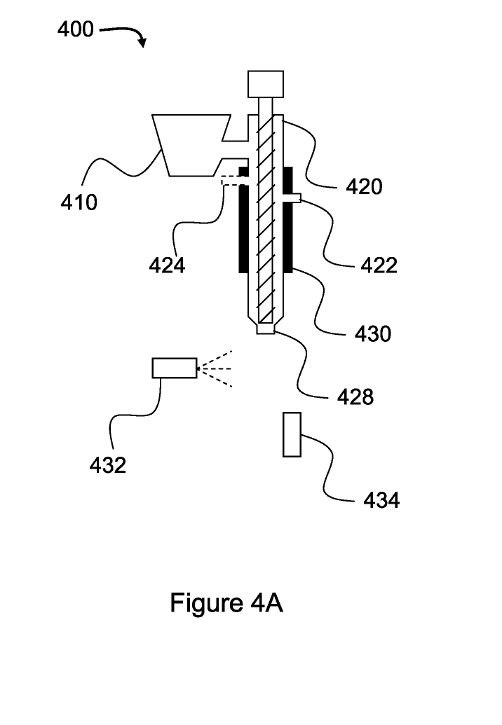

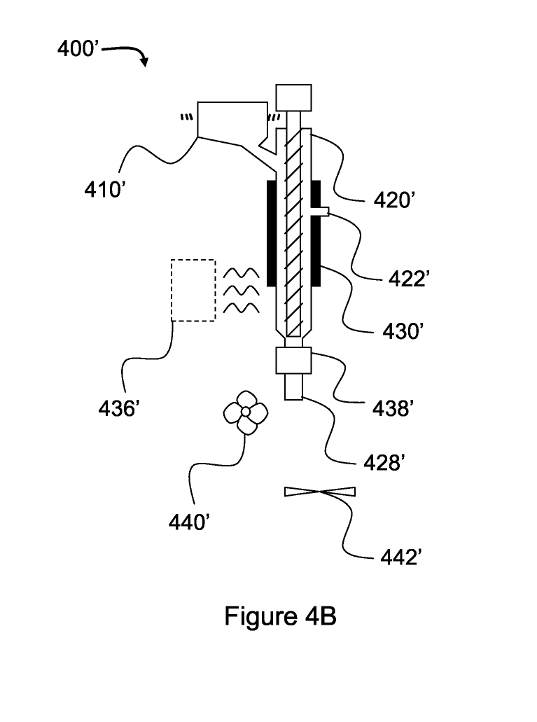

FIGS. 4A-B provide illustrations of nonlimiting examples of continuous systems according to the present invention. It should be noted that while FIGS. 4A-B depict vertical embodiments of continuous systems, continuous systems may be in any orientation relative to the ground. FIG. 4A provides a nonlimiting example of a continuous system 400 according to the present invention having a feeder 410 operably connected to an extruder 420, a VF-fluid (void forming fluid) inlet 422 operably attached to the extruder after the feeder 410, an agent inlet 424 operably connected to the extruder 420 between the feeder 410 and the VF-fluid inlet 422, heaters 430 along the extruder 420, an extrusion port 428 at the end of the extruder 420, a coating element 432 (illustrated as a sprayer) after the extrusion port 428, and a quality control element 434 after the coating element 432. In some embodiments, controlling the temperature (e.g., zonal temperature control) along the extruder may enable formation of a desired void space architecture.

FIG. 4B provides a nonlimiting example of a continuous system 400′ according to the present invention having a feeder 410′ (illustrated as being capable of vibrating) operably connected to an extruder 420′, a VF-fluid inlet 422′ operably attached to the extruder 420′ after the feeder 410′, heaters 430′ along the extruder 420′, a radiation source 436′ in radiative communication with the extruder 420′ (illustrated after the VF-fluid inlet 422′), pressure transducers 438′ near the end of the extruder 420′ to balance the pressure in the extruder 420′ with ambient conditions, an extrusion port 428′ (e.g., a die or a nozzle) at the end of the extruder 420′, and a cooling element 440′ (illustrated as a fan) after the extrusion port 428′, and a cutting element 442′ after the cooling element. In some embodiments, controlling the temperature (e.g., zonal temperature control) along the extruder and/or the pressure in the extruder may enable formation of a desired void space architecture.

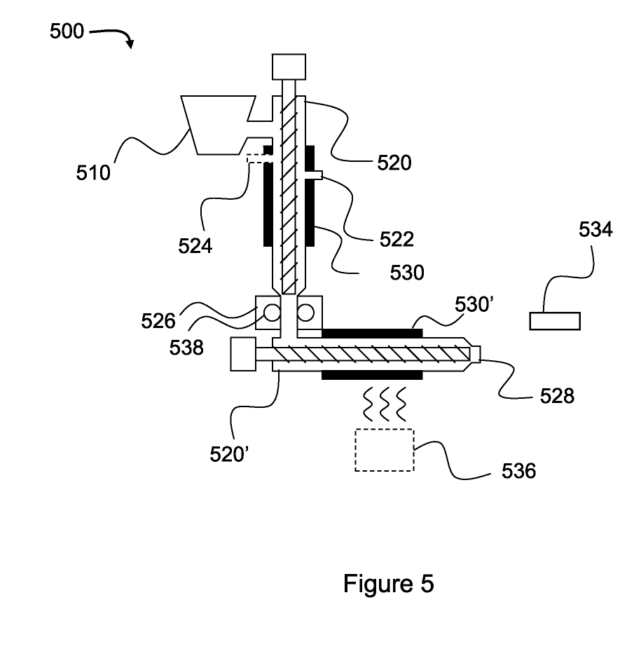

FIG. 5 provides an illustration of yet another nonlimiting example of a continuous system according to the present invention having two extruders 520 and 520′ operably connected so as to process essentially the same material. The second extruder 520′ may be advantageous to produce a more homogeneous polymer melt and/or void space architecture. FIG. 5 illustrates a system 500 having a feeder 510 operably connected to a first extruder 520, a VF-fluid inlet 522 disposed along the first extruder 520 after the feeder 510, an agent inlet 524 disposed along the first extruder 520 between the VF-fluid inlet 522 and the feeder 510, a second extruder 520′ operably connected to the end of the first extruder 520 with a gear pump 526 and pressure transducers 538 to assist in transfer of polymer melt from the first extruder 520 to the second extruder 520′ where the pressure in the first extruder 520 and the second extruder 520′ are different, heaters 530 and 530′ disposed along the first extruder 520 and the second extruder 520′, respectively (which in some embodiments may be at different temperatures), a radiation source 536 in radiative communication with the second extruder 520′, an extrusion port 528 at the end of the second extruder 520′, and a quality control element 534 after the extrusion port 528. In some embodiments with a multi-extruder system, controlling the temperature along and/or between each extruder and/or the pressure in each extruder may enable formation of a desired void space architecture.

In some embodiments, continuous systems of the present invention for forming controlled release vehicles of the present invention may include feeders operably connected to extruders and capable of feeding polymer pellets and/or polymer melts (including any agents or additives therein) to the extruder, heaters in thermal communication with at least a portion of the extruders, VF-fluid inlets operably connected to the extruders after the feeders, and extrusion ports at the end of the extruders. Optionally, continuous systems of the present invention for forming controlled release vehicles of the present invention may include equipment and/or areas for manipulating extrudates, partially crosslinking, additional inlets (e.g., to introduce agents), controlling pressure, cutting, coating, printing/imprinting, cooling, compression, monitoring the production parameters, quality control, and any combination thereof. The continuous systems of the present invention may, in some embodiments, advantageously reduce the number of handling steps, which for controlled release vehicles intended for applications involving humans and animals (e.g., tablets containing active pharmaceuticals) may reduce the potential for contamination.

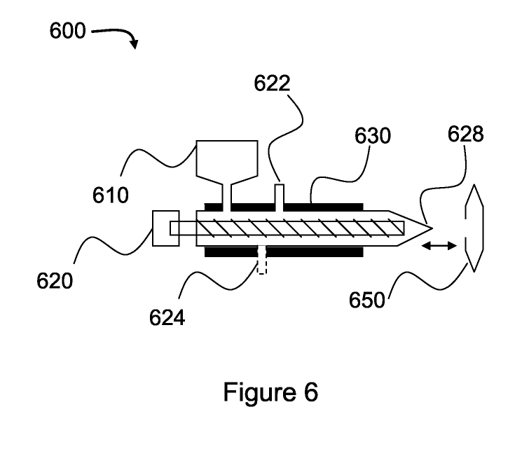

FIG. 6 provides an illustration of a nonlimiting example of a batch system 600 according to the present invention that includes a feeder 610 operably connected to an extruder 620, a VF-fluid inlet 622 operably attached to the extruder 620 after the feeder 610, an agent inlet 624 operably connected to the extruder 620 between the feeder 610 and the VF-fluid inlet 622, heaters 630 along the extruder 620, a extrusion port 628 at the end of the extruder 620, and a mold 650 capable of moving in and out of fluid communication with the extrusion port 628. It should be noted that while FIG. 6 depicts a horizontal embodiment of a batch system, batch systems may be in any orientation relative to the ground. In some embodiments, controlling the temperature and/or pressure along and/or in the extruder and/or of the mold may enable formation of a desired void space architecture. For example, at least one suitable system may be an injection molding system.

In some embodiments, batch systems of the present invention for forming controlled release vehicles of the present invention may include feeders operably connected to extruders, VF-fluid inlets operably attached to the extruders after the feeders, heaters along the extruder, extrusion ports at the end of the extruders, and molds capable of receiving polymer melt from the extrusion port such that the extruder is capable of injecting a desired volume of polymer melt into the molds. In some embodiments, the extrusion port may be operably connected to the mold. In some embodiments, the extruder may include a reciprocating screw to enable injection of a desired volume of polymer melt into molds. Optionally, batch systems of the present invention for forming controlled release vehicles of the present invention may include equipment and/or areas for partially crosslinking, additional inlets (e.g., to introduce agents), controlling pressure, cutting, coating, printing/imprinting, cooling, compression, monitoring production parameters, quality control, and any combination thereof. The batch systems of the present invention may, in some embodiments, be advantageous to form controlled release vehicles of substantially uniform size without additional processing steps like compression. Compression steps may, in some instances, negatively impact agents in controlled release vehicles, e.g., some active pharmaceuticals may decompose or react to inactive forms under pressure.

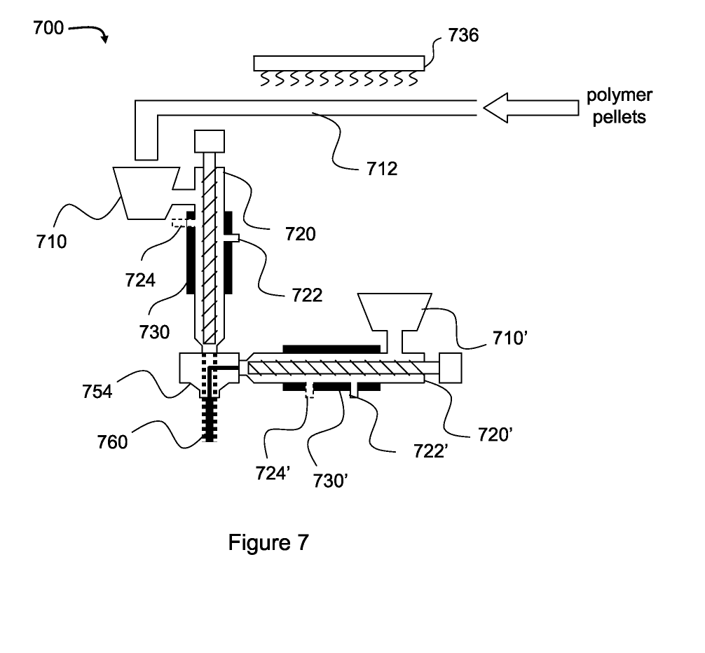

FIG. 7 provides a nonlimiting illustration of a continuous coextrusion system 700 according to the present invention that includes (1) a first feeder 710 operably connected to a first extruder 720, heaters 730 along the first extruder 720, a first VF-fluid inlet 722 operably attached to the first extruder 720 after the first feeder 710, and a first agent inlet 724 operably connected to the first extruder 720 between the first feeder 710 and the first VF-fluid inlet 722; (2) a pellet transportation system 712 that brings polymer pellets into radiative communication with a radiation source 736 (e.g., an electron beam) and transports the radiated polymer pellets to the first feeder 710 that is operably connected to the first extruder 720; (3) a second feeder 710′ operably connected to a second extruder 720′, heaters 730′ along the first extruder 720′, a second VF-fluid inlet 722′ operably attached to the second extruder 720′ after the second feeder 710′, and a second agent inlet 724′ operably connected to the second extruder 720′ after the second VF-fluid inlet 722′; and (4) a coextruder 754 operably connected to the first extruder 720 and the second extruder 720′ where the coextruder 754 is configured to direct the polymer melt from each extruder to form a desired complex macrostructure in the extrudate 760 (as generally depicted in FIG. 7, a core-sheath macrostructure, e.g., a layer disposed on at least a portion of the surface of the polymeric matrix). In some embodiments, where the sheath is a non-foamed surface layer disposed on at least a portion of the surface of the core (i.e., a foamed polymeric matrix), the second extruder as depicted in the nonlimiting example of FIG. 7 may not include a VF-fluid inlet. In some embodiments with a co-extrusion system, controlling the temperature along of each extruder and/or the pressure in each extruder may enable formation of a desired void space architecture.

In some embodiments, forming a desired void space architecture in a polymer matrix of controlled release vehicles of the present invention may involve (1) introducing a void forming fluid (“VF-fluid”) into a polymer melt, (2) nucleating voids, and (3) growing voids. It should be noted that systems may be designed to, in some embodiments, provide the appropriate amount of time for each of these mechanisms to occur. Accordingly, in some embodiments, nucleation may be significantly fast so as to appear that growth occurs immediately after introduction of the VF-fluids.

In some embodiments, a polymer melt to which VF-fluids are introduced may be at an elevated pressure. Pressures suitable for a polymer melt to which VF-fluids are added may, in some embodiments, range from a lower limit of about 500 psi, 750 psi, 1000 psi, or 1500 psi to an upper limit of about 3000 psi, 2500 psi, 2000 psi, or 1500 psi, and wherein the pressure of the polymer melt may range from any lower limit to any upper limit and encompass any subset therebetween.

Temperatures suitable for a polymer melt to which VF-fluids are added may, in some embodiments, be from at or above the melting point to about the degradation point of the polymeric components of the polymer melt (e.g., ethylene copolymers, ethyl celluloses, thermoplastic polyurethanes, and/or additional thermoplastic polymers). For example, temperatures suitable for polymer melt to which VF-fluids are added may, in some embodiments, range from a lower limit of about 50° C., 60° C., 75° C., 100° C., or 125° C. to an upper limit of about 500° C., 400° C., 350° C., 300° C., 250° C., 225° C., 200° C., 175° C., or 150° C., and wherein the temperature may range from any lower limit to any upper limit and encompass any subset therebetween. Temperature selection may, in some embodiments, depend on, inter alia, the presence and composition of agents, optional additives, and/or optional additional ingredients, and the location and introduction method thereof so as to minimize thermal degradation thereof.

VF-fluids suitable for forming a desired void architecture according to some embodiments of the present invention may include, but are not limited to, air, an inert gas (e.g., helium, nitrogen, argon, carbon dioxide, n-butane, or isobutane), volatile liquids (e.g., water, methanol, or acetone), hydrocarbons (e.g., butane, isobutane, or pentane), halogenated hydrocarbons, perfluorocarbons, and the like, or any mixture thereof. In some embodiments, the VF-fluids may be in a gas, liquid, subcritical, or supercritical form dissolved in the polymer melt. In some embodiments of the present invention, VF-fluids may serve to form the void space architecture and as an agent, e.g., a perfluorocarbon gas that provides contrast in ultrasound imaging. In some embodiments of the present invention, VF-fluids may be a volatile liquid that serves to form the void space architecture and plasticize the polymer melt.

In some embodiments, the amount of VF-fluids added to a polymer melt may be at or below the saturation point of the VF-fluids in the polymer melt.

The parameters of introducing VF-fluids (gas and/or liquid) into the polymer melt may be controlled to provide control over the diameter distribution of the pores of the resultant controlled release vehicle of the present invention.

Suitable parameters to adjust may include, but are not limited to, temperature of the polymer melt, temperature of the VF-fluid, pressure of the VF-fluid, composition of the VF-fluid, composition of the polymer melt, pressure of the polymer melt, degree of partially crosslinking of the polymer melt, optional partially crosslinking during and/or after pore formation, temperature of the die, speed of the screw rotation, geometry of the screw, and any combination thereof. In some embodiments, methods may involve introducing VF-fluids into a polymer melt and allowing time to pass to allow for the VF-fluids to disperse at least substantially-homogeneously throughout the polymer melt.

Nucleation of voids may, in some embodiments, involve reducing the temperature and/or pressure of the polymer melt having VF-fluids therein. In some embodiments, void nucleation may occur at a temperature ranging from the melting point of the polymer melt to the temperature at which fluid was introduced into the polymer melt. In some embodiments, nucleation of voids may occur at a temperature of less than about 50% lower than the temperature at which fluid was introduced into the polymer melt, less than about 25% lower, or less than about 10% lower.

In some embodiments, nucleation of voids may occur at a pressure ranging from about ambient to about the pressure at which fluid was introduced into the polymer melt. In some embodiments, nucleation of voids may occur at a pressure ranging from a lower limit of about ambient, 25 psi, 250 psi, 500 psi, 750 psi, 1000 psi, or 1500 psi to an upper limit of about 3000 psi, 2500 psi, 2000 psi, 1500 psi, or 1000 psi, and wherein the pressure of the polymer melt may range from any lower limit to any upper limit and encompass any subset therebetween.

Growth of voids may, in some embodiments, involve increasing temperature and/or reducing pressure of the polymer melt having nucleated voids. In some embodiments, growth of voids may occur at a temperature above the temperature of void nucleation, including temperatures above the temperature at which fluid was introduced into the polymer melt. In some embodiments, void growth may occur at a temperature of at least about 10% greater than the temperature of void nucleation, at least about 50% greater, at least about 100% greater, or at least about 150% greater. In some embodiments, void growth may occur at a temperature of at least about 5% greater than the temperature at which fluid was introduced into the polymer melt, at least about 10% greater, or at least about 25% greater.

In some embodiments, growth of voids may occur at a pressure ranging from about ambient to about the pressure at which fluid was introduced into the polymer melt. In some embodiments, void growth may occur at a pressure ranging from a lower limit of about ambient, 25 psi, 250 psi, 500 psi, 750 psi, 1000 psi, or 1500 psi to an upper limit of about 3000 psi, 2500 psi, 2000 psi, 1500 psi, or 1000 psi, and wherein the pressure of the polymer melt may range from any lower limit to any upper limit and encompass any subset therebetween.

As eluted to above, systems of the present invention may, in some embodiments, be capable of having temperature control so as to allow for introduction of VF-fluids and nucleation in the same system. Systems of the present invention may, in some embodiments, comprise at least one extruder having different temperature zones. In some embodiments, systems of the present invention may comprise multiple extruders having independent temperatures and/or temperature zones.

Forming controlled release vehicles of the present invention having a complex macrostructure may involve coextrusion from at least two polymer melts. Systems of the present invention for forming complex macrostructures of controlled release vehicles of the present invention may include systems (and components thereof) similar to those described above in FIGS. 4-6 modified so as to feed into a coextruder that directs the extrusion to form the desired macrostructure.

In some embodiments of the present invention with controlled release vehicles having agents, incorporation of the at least one agent may be at many points along the production of the controlled release vehicle. Some embodiments of the present invention may involve forming controlled release vehicles of the present invention from a polymer melt comprising ethylene copolymers, ethyl celluloses, and/or thermoplastic polyurethanes and agents. In some embodiments, a polymer melt comprising ethylene copolymers, ethyl celluloses, and/or thermoplastic polyurethanes and agents may be achieved by the addition of the agents to the ethylene copolymers, ethyl celluloses, and/or thermoplastic polyurethanes while the ethylene copolymers, ethyl celluloses, and/or thermoplastic polyurethanes are in polymer melt form (e.g., a polymer melt in the feeder or a polymer melt in the extruder). In some embodiments, a polymer melt comprising ethylene copolymers, ethyl celluloses, and/or thermoplastic polyurethanes and agents may be achieved by the addition of the agents to the ethylene copolymers, ethyl celluloses, and/or thermoplastic polyurethanes while the ethylene copolymers, ethyl celluloses, and/or thermoplastic polyurethanes are in solid or semi-solid form (e.g., polymer pellets, flake, and/or powder in the feeder to be melted).

Some embodiments of the present invention may involve introducing agents into the polymer melt while in the extruder of a system of the present invention during the formation of controlled release vehicles of the present invention, e.g., through the agent inlet described above. The introduction may be of the agent to the polymer melt while the polymer melt is in the extruder may advantageously reduce the heat history of the agent, which may be particularly advantageous for agents like some active pharmaceuticals that have a susceptibility to thermal degradation.

Some embodiments of the present invention may involve loading the controlled release vehicles with agents after forming the controlled release vehicles. Loading agents into already formed controlled release vehicles may include, but are not limited to, causing the agents to be absorbed into the polymeric matrix and/or void space architecture, which may include prolonged soaking in a fluid (e.g., supercritical CO₂, an alcohol, or the like) comprising agents, increasing temperature and pressure to facilitate absorption, and the like. Loading after formation may advantageously provide loading near the outer surface of the controlled release vehicle, which may provide a release profile with an initial bolus. Further, loading after formation may, in some embodiments, be advantageous for certain agents that are temperature sensitive, like some biological compounds and cells. By way of nonlimiting example, a controlled release vehicle may be produced with a void space architecture suitable for loading stem cells because of size and temperature considerations, inter alia, relating to stem cells.

It should be noted that in some embodiments, agents may be incorporated into the controlled release vehicles of the present invention in any combination of addition to the polymer pellets (or the like) and/or polymer melt in the feeder, introduction into the extruder via a feeder separate from the polymer pellet (or the like) and/or polymer melt feeder, introduction into the polymer melt while in the extruder, and loading after formation of the controlled release vehicle.

It should be noted that the additional elements above (e.g., additional thermoplastic polymers, plasticizers, and/or additional ingredients) may be incorporated into the controlled release vehicles in methods similar to those described for agents. One skilled in the art should understand the appropriate incorporation method based on the additional element being added and the desired controlled release vehicle being produced. By way of nonlimiting example, additional thermoplastic polymers may be most effectively incorporated into the formation of controlled release vehicles at the polymer pellet (or the like) and/or polymer melt stages.

Suitable equipment and/or areas for partially crosslinking areas in systems of the present invention (continuous or batch) may include, but are not limited to, radiation sources that induce partial crosslinking of at least a portion of the polymer pellets (or the like) and/or the polymer melt (e.g., electron beams, high-energy ionizing radiation, gamma radiation, x-ray radiation, UV light, and the like, and combinations thereo), autoclaves and/or steam tubes to induce partial crosslinking of at least a portion of the polymer pellets (or the like) and/or the polymer melt, or additional inlets to introduce a chemical crosslinker (e.g., initiators, free radical generators, peroxides, or dicumyl peroxide). In some embodiments, multiple partially crosslinking methods and/or equipments may be used. By way of nonlimiting example, a peroxide may be used to initiate partially crosslinking in the extruder and a radiation source or autoclave may be used after extrusion (on injection into a mold) to complete partially crosslinking.

In some embodiments, non-chemical partially crosslinking methods may be used so as to (1) minimize additives in the resultant controlled release vehicles of the present invention and (2) mitigate the exposure of an agent to a chemical crosslinker that may negatively impact the agent (e.g., a peroxide). In some embodiments, a radiation dose (e.g., from an electron beam or other suitable source) ranging from a lower limit of about 1 mGy, 10 mGy, 100 mGy, 1 Gy, 10 Gy, 100 Gy, 1 kGy, 2 kGy, or 5 kGy to an upper limit of about 50 kGy, 40 kGy, 30 kGy, 20 kGy, 15 kGy, 10 kGy, 5 kGy, 1 kGy, 100 Gy, 10 Gy, or 1 Gy may be used as a nonchemical partially crosslinking method, and wherein the radiation dose may range from any lower limit to any upper limit and encompass any subset therebetween.

Without being limited by theory, it is believed that partially crosslinking (chemical and/or non-chemical) may decrease the melt-flow index of the polymer melt, which in turn, may affect the void space architecture and controlled release properties of the polymeric matrix. For example, decreasing the melt flow index may enable formation of a void space. Further, increasing partially crosslinking may retard the release rate of a polymeric matrix. Accordingly, partially crosslinking (chemical and/or non-chemical) may, in some embodiments be controlled. In some embodiments, the extent of partially crosslinking may be such that the melt flow index decreases by as much as 99%, more preferably about 10% to about 95%, or most preferably about 25% to 90%, including any subset therebetween. It should be noted that additional ingredients and/or additives may be utilized to achieve a decrease in melt-flow index. For example, lecithin may be utilized with ethylene vinyl acetate copolymers to reduce the melt-flow index.

Crosslinking areas, in some embodiments, may be advantageous to control the rate of formation of the voids and/or pores, thereby controlling the void space architecture (including the parameters discussed herein). Crosslinking areas, in some embodiments, may be advantageous to control, and in some embodiments, substantially stop the formation (e.g., growth) of the voids and/or pores, thereby controlling the void space architecture (including the parameters discussed herein). Crosslinking areas may, in some embodiments, be at any point along the extruder and preferably after the VF-fluid inlet port. One skilled in the art with the benefit of this disclosure should understand that the extruder may need to be engineered to allow for radiation to reach the polymer melt within the extruder. For example, an extruder may comprise a port, a window, or the like to allow for homogenous irradiation of a polymer melt therein.

Some embodiments may involve partially crosslinking a polymer melt or precursor thereof (e.g., polymer pellets or the like) before introduction into the extruder during the production of controlled release vehicles of the present invention. Some embodiments may involve partially crosslinking polymer pellets (or the like) at a different location than where extrusion occurs. Some embodiments may involve partially crosslinking a polymer melt while in the extruder during the production of controlled release vehicles of the present invention. Some embodiments may involve partially crosslinking a polymer melt after extrusion during the production of controlled release vehicles of the present invention. Some embodiments may involve partially crosslinking a polymer melt after injection into a mold during the production of controlled release vehicles of the present invention. Some embodiments may involve multiple partially crosslinking steps during the production of controlled release vehicles of the present invention.

Suitable equipment and/or areas for manipulating extrudates in systems of the present invention may be operably connected to the extruder so as to assist in the continuous removal of the extrudate from the extruder. By way of nonlimiting example, an extrudate may be manipulated by a roller, a series of rollers, a pulling system, a strand pelletizer, winding spools, or the like.

Suitable equipment and/or areas for cutting in systems of the present invention may be operably connected to the extruder so as to section the extrudate (product from the extruder) as it leaves the extruder or at some predetermined point after the extruder. By way of nonlimiting example, an extrudate from a continuous system may be transported by conveyor to cool before cutting. Where desirable, some embodiments may involve cutting extrudates and/or molds during the production of controlled release vehicles of the present invention.

Suitable equipment and/or areas for coating in systems of the present invention may be capable of coating the extrudate (before or after cooling) or coating the controlled release vehicle after cutting and/or removal from a mold. Suitable coating methods may include, but are not limited to, spraying, drizzling, showering, sputtering, passing through liquid (e.g., in a bath), passing through a vapor and/or mist, any hybrid thereof, and any combination thereof. Suitable coatings for use in conjunction with the present invention may include, but are not limited to, coatings that protect the controlled release vehicle, at least in part, from gastric juices, photo-induced degradation, bacterial or fungal contamination, environmental degradation, and the like, and any combination thereof. Some embodiments may involve coating extrudates and/or controlled release vehicles of the present invention.

Suitable equipment and/or areas for printing/imprinting in systems of the present invention may be capable of printing on the extrudate (before or after cooling) or printing on the controlled release vehicle after cutting and/or removal from a mold. Printing and/or imprinting may, in some embodiments, enable information to be printed and/or imprinted directly on controlled release vehicles of the present invention. Information may be printed and/or imprinted, in some embodiments, in the form of lines, shapes, symbols, letters, bar-codes, 2-D codes, and the like, and any combination thereof. Information suitable for printing and/or imprinting may include, but is not limited to, manufacture identification, agent identification, manufacturing information (e.g., date, time, and/or parameters of production), lot identification, production line identification, and any combination thereof. By way of nonlimiting example, in continuous systems, printing and/or imprinting the production line and date of manufacturing may, in some embodiments, advantageously provide manufacturers a method of identifying and/or authenticating controlled release vehicles of the present invention after distribution. In some embodiments, the information printed and/or imprinted on a controlled release vehicle of the present invention may be readable by devices, e.g., by laser scanning, taking pictures (e.g., with a mobile device), and the like.

Suitable equipment and/or areas for cooling in systems of the present invention may be capable of cooling the extrudate (before or after cutting and/or coating) or the controlled release vehicle in or out of the mold after cutting and/or coating. Cooling may be passive (e.g., allowing to cool in ambient conditions) or active (e.g., with moving air, with moving liquid, in a cooled environment, or the like). Some embodiments may involve cooling extrudates and/or molds during the production of controlled release vehicles of the present invention.

Suitable equipment and/or areas for monitoring the production parameters in systems of the present invention may be capable of monitoring parameters like feeder temperature, feeder calibration, feeder rate, extruder temperature, extruder pressure, extruder water discharge flow rate (generally related to extruder temperature), extruder's screw speed, extruder motor amperages, extruder motor torque, mass flow rate of material exiting the extruder, transfer of material from a first extruder to a second extruder, VF-fluid inlet pressure, VF-fluid inlet flow rate, VF-fluid inlet temperature, agent inlet pressure, agent inlet flow rate, agent inlet temperature, pressure at the die, partially crosslinking element strength (e.g., strength of an electron beam, which can be measured in gray), temperature and/or pressure of partially crosslinking elements (e.g., autoclaves), print geometry, print quality (e.g., ink density), and print information, roller pressure, roller draw rate/speed, air flow in cooling areas, water bath cooling temperatures, coating temperature, coating flow rate, cutter speed, cutter temperature, parameters of equipment operably connected to the system (e.g., pumps, gears, and the like), and any combination thereof. Some embodiments may involve monitoring the production parameters of the systems for producing controlled release vehicles of the present invention.

Suitable equipment and/or areas for quality control in systems of the present invention may be capable of analyzing the products from the continuous or batch systems (e.g., the extrudate and the molded controlled release vehicles). In some embodiments, quality control may be qualitative or quantitative. Quality control may, in some embodiments, analyze aspects of the void space architecture (e.g., void space volume and void diameter), composition of agents (e.g., any degree of decomposition or polymerization), crystallinity of agents, concentration of agents, purity of agents, presence of contaminants, composition of contaminants, concentration of contaminants, composition of the polymeric matrix, crystallinity of the polymeric matrix, and the like, and any combination thereof. Examples of techniques that may, in some embodiments, be employed in equipment and/or areas for quality control for use in conjunction with the present invention may include, but are not limited to, magnetic resonance imaging, computer tomography (CT), ultrasound, near-infrared spectroscopy, Raman spectroscopy, Fourier transform-infrared (FT-IR) spectroscopy, and the like. By way of nonlimiting example, an extrudate may pass through a CT scanner to determine the void space volume of the controlled release vehicle and pass through an FT-IR spectrometer to detect degradation of the agent. Some embodiments may involve performing quality control measurements during the production of controlled release vehicles of the present invention.

In some embodiments, predetermined limits may be placed on production parameters and/or product quality. If the production parameters and/or product quality deviate outside the predetermined limits, the system (or components thereof) may, in some embodiments, provide feedback, trigger an alarm (local and/or remote), send a message to person (e.g., via email, text, or page), take self-correcting measures, divert product to another area for further analysis, shutdown production or some portion thereof, and any combination thereof. By way of nonlimiting example, in the production of a controlled release vehicle having a temperature-sensitive, active pharmaceutical, systems may monitor the temperature of the extruder in several locations, have a narrow temperature window, and divert product from the production line to a holding bin for further analysis if the temperature at just one location along the extruder is outside the temperature window. By way of another nonlimiting example, in the production of a controlled release vehicle having a temperature-sensitive active pharmaceutical, systems may monitor the product for degradation of the active pharmaceutical and shutdown the system when degradation, e.g., due to thermal degradation, is observed above a certain level.

In some embodiments, the controlled release vehicles of the present invention may be in the form of a film, a sheet, a fiber, a filament, a ribbon, a band, a rod, a sphere, a pellet, a tablet, a discus, an organ-shape, a hollow tube-shape, a ring, a trapezoidal shape, a polygonal shape, and the like, any form substantially similar to a form thereof, or any hybrid thereof. One skilled in the art should understand how to modify the systems and methods of forming to achieve the forms, having the benefit of this disclosure. By way of nonlimiting example, a patch may advantageously have a controlled release vehicle in a film form. In some embodiments, the film may be formed by extrusion onto a conveyer with an appropriately shaped die, which may optionally include a puller system or rollers to create a desired thickness. In some embodiments, the film may be formed by blowing methods, compression molding methods, and the like.

It should be noted that while the description provided herein generally refers to systems for producing controlled release vehicles, in some embodiments, the various components of the systems described herein may be combined into apparatuses.

Systems and/or apparatuses for producing controlled release vehicles (according to any embodiments described herein) may, in some embodiments, include at least one extruder with at least one extrusion port (e.g., a die or a nozzle) and at least one VF-fluid inlet port. Optionally, systems and/or apparatuses for producing controlled release vehicles (according to any embodiments described herein) may further include (individually or in any combination) at least one feeder, at least one agent inlet, at least one heater, at least one mold, at least one element and/or area for partially crosslinking, at least one element and/or area for coating, at least one element and/or area for printing/imprinting, at least one element and/or area for cooling, at least one element and/or area for cutting, at least one element and/or area for manipulating extrudates, at least one element and/or area for monitoring production parameters, and at least one element and/or area for quality control.

### IV. Implementing Controlled Release Vehicles

In some embodiments, the controlled release vehicles of the present invention may release agents with a desired release profile. The release profile may include, but is not limited to, release at a constant rate (e.g., zero order being diffusion controlled), a sustained rate, an exponentially increasing rate, an exponentially decreasing rate, a first order decaying rate, a rate decreasing with the square root of time (e.g., monolithic devices), a bolus release, any hybrid thereof, and any combination thereof.

In some embodiments, the controlled release vehicles of the present invention may reduce the concentration of a constituent in a fluid with a desired uptake profile. The uptake profile may include, but is not limited to, uptake at a constant rate, a sustained rate, an exponentially increasing rate, an exponentially decreasing rate, a first order decaying rate, a rate decreasing with the square root of time, a bolus uptake (i.e., quick uptake to saturation of the agent), any hybrid thereof, and any combination thereof.

One skilled in the art, with the benefit of this disclosure, should understand that the release and/or uptake profiles of the controlled release vehicles of the present invention depend, inter alia, on the void space architecture, the composition of the polymeric matrix, the size and shape of the controlled release vehicles, and the size and shape of the agents.

In some embodiments, the controlled release vehicles of the present invention may be designed to release two or more agents at different rates. By way of nonlimiting example, bimodal void diameter distributions may be employed in controlled release vehicles of the present invention to achieve release of two or more agents at different rates. By way of another nonlimiting example, a narrow void diameter distribution, e.g., a void diameter distribution having a full width at half max of about 20% or less of the average void diameter, may allow for different release rates for two or more agents having different molecular weights, sizes, and/or shapes. The nonlimiting example may be extended to an average void diameter, void distance distributions, an average void distance, pore diameter distributions, and average pore diameters. By way of nonlimiting yet another example, a single controlled release vehicle may include two agents with the first having a molecular weight less than about 1,000 amu and the second having a molecular weight greater than about 10,000 amu. With a smaller average pore diameter, the lower molecular weight agent may be able to traverse the pores while the larger molecular weight may have to diffuse through portions of the polymeric matrix to be released. This nonlimiting example may be extended to agents having differing sizes and shapes, or other differing characteristics, not just molecular weight.

In some embodiments, the controlled release vehicles of the present invention may be multi-acting vehicles. As used herein the term “multi-acting” refers to serving at least two purposes, e.g., providing tracking of the vehicle, releasing agents in a controlled manner, and removing constituents from a fluid. In some embodiments, the controlled release vehicles of the present invention may comprise at least one active agent, at least one removal agent, and a polymeric matrix having a void space architecture. In some embodiments, the controlled release vehicles of the present invention may comprise at least one active agent, at least one tracking agent, and a polymeric matrix having a void space architecture. In some embodiments, the controlled release vehicles of the present invention may comprise at least one removal agent, at least one active agent, at least one tracking agent, and a polymeric matrix having a void space architecture. The embodiments extend to complex macrostructure embodiments.

In some embodiments, the controlled release vehicles of the present invention may be administered to a patient. As used herein, the term “subject” and “patient” are used interchangeably herein and refer to both human and nonhuman animals and insects. The term “nonhuman animals” as used herein includes all vertebrates, e.g., mammals and non-mammals, such as nonhuman primates, mice, rats, sheep, dogs, cats, horses, cows, chickens, amphibians, fish, reptiles, and the like. The term “insects” as used herein includes all arthropods, e.g., bees, flies, Drosophila flies, beetles, spiders, and the like.

In some embodiments, the controlled release vehicles of the present invention may be administered to patients orally (e.g., pills, tablets, and the like), subdermally (e.g., subdermal implants), transdermally (e.g., patches, lotions, cosmetics, and the like), transmucosally (e.g., oromucosal inserts, intrauterine devices, intravaginal rings, dental fibers, and the like), and/or as a part of an implantable medical device. In some embodiments, agents in controlled release vehicles of the present invention may be administered to patients by oral delivery of the controlled release vehicle, subdermal implantation or injection of the controlled release vehicle, placement of the controlled release vehicle for transdermal administration of the agent, and/or implanting a medical device including a controlled release vehicle of the present invention.

In some embodiments, the controlled release vehicles of the present invention may be for the prevention, mitigation, and/or treatment of diseases, conditions, and/or symptoms thereof in a patient. By way of nonlimiting example, the controlled release vehicles of the present invention may include agents that slow the progression of HIV to AIDS. Slowing the progression may require several agents with different release profiles to be most effective, which is where the complex macrostructures of the present invention may be advantageously applicable. By way of another nonlimiting example, a patch comprising controlled release vehicles of the present invention having antioxidants therein may advantageously be applicable to patients exposed to low-dose, long-term radiation, e.g., astronauts in long-term space flight, to mitigate the effect of the radiation on the patient's systems, e.g., cardiovascular and gastrointestinal systems. By way of another nonlimiting example, a stent comprising a controlled release vehicle of the present invention having stem cells and stem cell factors towards cardiac epithelial cells therein may be advantageous in directing stem cell differentiation to cardiac epithelial cells where the controlled release of stem cell factors perhaps provides a prolonged dose that enhances the desired differentiation.

In some embodiments, the controlled release vehicles of the present invention may be a component of a kit for the treatment or prevention of a disease or condition in a patient. In some embodiments, a kit may include a set of instructions and at least one controlled release vehicle of the present invention. In some embodiments, a kit may include a set of instructions and an article comprising at least one controlled release vehicle of the present invention. By way of nonlimiting example, a kit for treating multidrug-resistant cancers may include a set of instructions and a controlled release vehicle of the present invention as a tablet having a complex macrostructure that releases doxorubicin to treat the cancer and siRNA to suppress the cellular-resistance to treatment.

In some embodiments, the controlled release vehicles of the present invention may be an implant, or component thereof, for a patient, e.g., in vivo implants, subdermal implants, intramuscular implants, mucosal implants, or ocular implants. By way of nonlimiting example, a controlled release vehicle of the present invention may be predominant composition of an ocular implant where the controlled release vehicle includes brimonidine to treat ocular hypertension.

In some embodiments, the controlled release vehicles of the present invention may be a part of another article or composition. The articles or compositions may be for medical products (e.g., bandages, wound dressings, transdermal patches, medical implants, contraceptive devices, bags and containers for storing and/or transporting bodily fluids, or filters), personal care products (e.g., lotions, creams, cosmetics, lipsticks and lip glosses, cleansing patches, and feminine hygiene products), consumer products (e.g., filters or components thereof, woven or nonwoven materials, laminated articles, food containers, or bags), fragrant products (e.g., perfumes, deodorizers, and scented oils), fertilizers, smoking devices, chemical and biological release devices, and the like.

In some embodiments, the controlled release vehicles of the present invention may be useful as additives in consumer health and beauty products, e.g., deodorants, perfumes, cosmetics, lotions, creams, lip glosses, and the like.

In some embodiments, the agents of controlled release vehicles of the present invention may be administered to a patient without administration of the controlled release vehicle to the patient. By way of nonlimiting example, insect repellents may be released from a controlled release vehicle so as to administer the insect repellant to the insect without the insect having to come in contact with the controlled release vehicle. Further, administration to a patient may include, in some embodiments, spraying and/or aerosolizing the controlled release vehicles. In some embodiments, controlled release vehicles may be heated to hasten release of agents therein, e.g., including as a part of a candle or oil to be burned.

In some embodiments, the controlled release vehicles of the present invention may be useful in the administration of agents to or remove contaminants from the environment. By way of nonlimiting example, the controlled release vehicles of the present invention may be included in soils or fertilizers so as to provide an agent useful for plant growth or parasite prevention. By way of another nonlimiting example, controlled release vehicles may be included in articles like filters or geotextiles so as to remove contaminants from the environment, e.g., heavy metals or toxins.

In some embodiments, the controlled release vehicles of the present invention may be useful in the preservation of articles disposed in containers, e.g., bodily fluids, food, and clothing. In some embodiments, the controlled release vehicles of the present invention may be incorporated in the material of a container and/or as at least a portion of a coating on the interior of the container. Examples of suitable containers may include, but are not limited to, bags, blood bags, boxes, plastic containers, and the like. By way of nonlimiting example, controlled release vehicles with agents capable of absorbing moisture and agents capable of inhibiting fungi growth may be advantageous for food preservation and be placed in a container as a lining or internal coating of the container or as an integral part of the container. By way of another nonlimiting example, containers containing controlled release vehicles with agents like juniper essential oils to mitigate moth infestation.

In some embodiments, the controlled release vehicles of the present invention may be useful in the release of agents having an affect on the olfactory system. By way of nonlimiting example, food packaging may contain controlled release vehicles of the present invention with compounds that provide an inviting smell without having opened the food packaging.

In some embodiments, the controlled release vehicles of the present invention may be useful in the release of agents having a flavor, which may be useful in flavoring foods, liquids, and gas streams. By way of nonlimiting example, the controlled release vehicles of the present invention having agents like menthol may be incorporated into smoking device filters or sections thereof. By way of nonlimiting example, the controlled release vehicles of the present invention having agents for removing harmful components of smoke streams may be incorporated into smoking device filters or sections thereof.

### V. Agents

Suitable agents for use in conjunction with the present invention may, in some embodiments, be for the prevention, mitigation, and/or treatment of diseases, conditions, and/or symptoms thereof in a patient. Examples of diseases and conditions may include, but are not limited to, arthritis, rheumatoid arthritis, juvenile rheumatoid arthritis, psoriatic arthritis, osteoarthritis, gouty arthritis, refractory rheumatoid arthritis, chronic non-rheumatoid arthritis, osteoporosis/bone resorption, osteophorosis, ulcerative colitis, skin diseases, psoriasis, acne vulgaris, rosacea, dermatitis, contact dermatitis, eczema, delayed-type hypersensitivity in skin disorders, type I diabetes, type II diabetes, Alzheimer's disease, inflammatory disorders, immunodeficiency, inflammatory bowel disease, irritable bowel syndrome, Crohn's disease, diarrhea disease, antibiotic associated diarrhea, pediatric diarrhea, chronic constipation, heartburn, appendicitis, autoimmune disorders, multiple sclerosis, muscle degeneration, coeliac disease, diabetes mellitus, organ transplantation, bacterial infections, viral infections, fungal infections, periodontal disease, urogenital disease, sexually transmitted disease, HIV infection, HIV replication, HIV associated diarrhea, surgical associated trauma, surgical-induced metastatic disease, nausea, weight loss, weight gain, anorexia, bulimia, fever control, cachexia, wound healing, ulcers, gut barrier function, allergies, Hay Fever, allergic rhinitis, anaphylaxis, asthma, respiratory disorders, lung diseases, pulmonary fibrosis, chronic obstructive pulmonary disease, circulatory disorders, anemia, disorders of the blood coagulation system, renal disease, disorders of the central nervous system, hepatic disease, ischemia, nutritional disorders, endocrine disorders, epidermal disorders, multiple myeloma, uveititis, acute and chronic myelogenous leukemia, anti-clotting, coronary heart disease, vasculitis, ischemic heart disease, atherosclerosis, strokes, peripheral arterial disease, ischemic-induced cell damage, high blood cholesterol levels, high-density lipoprotein (HDL) levels, high blood pressure, pancreatic 13 cell destruction, rheumatoid spondylitis, adult respiratory distress syndrome (ARDS), bone resorption diseases, ischemia reperfusion injury, brain trauma, cerebral malaria, sepsis, septic shock, toxic shock syndrome, blood infection, fever, myalgias due to infection, HIV-1, HIV-2, HIV-3, immune system disorders, cytomegalovirus, colds, influenza, adenovirus, the herpes viruses (including HSV-1, HSV-2), herpes zoster infection, herpes simplex/cold sores, infections, disorders associated with C-reactive protein, myositis, lupus, Celiac disease, prostatitis, tumor, sexual dysfunction, inflammatory disease, thyroid diseases, pregnancy, headaches, acute pain, rashes, addiction, addiction to habit forming drugs, addiction to smoking, upper respiratory tract infection, neurodegenerative disease, dyslexia, dyspraxia, autism, Asperger's disease, mild cognitive impairment, poor concentration, attention deficit disorder (ADD), attention deficit hyperactive disorder (ADHD), depression, mood swings, bipolar disorders, cancer, leukemia, acute and chronic myelogenous leukemia, colon cancer, prostate cancer, kidney cancer, liver cancer, breast cancer, lung cancer, melanoma, brain cancer, cervical cancer, Hodgkin's lymphoma, non-Hodgkin's lymphoma, ovarian cancer, testicular cancer, thyroid cancer, uterine cancer, urinary tract infection, nervous system infection, and the like. The controlled release vehicles of the present invention may be useful in the prevention, mitigation, and/or treatment of other diseases, conditions, and/or symptoms.

Nonlimiting examples of agents suitable for use in conjunction with the present invention may include, but are not limited to, active pharmaceuticals, prodrugs of active pharmaceuticals, active biologicals, antibiotics, antifungals, cells and cell-like structures, antitoxins, antigens, therapeutics, preventive therapeutics, nutritional supplements, imaging agents, fluid stabilizers, food agents, flavorants, olfactory agents, plant agents, chemical-reaction agents, and any combination thereof. It should be noted that agents may overlap into two or more types of suitable agents.

Examples of suitable agents (active agents (e.g., active pharmaceuticals and prodrugs of active pharmaceuticals), removal agents, and tracking agents) for use in conjunction with the present invention may include, but are not limited to, 16-alpha fluoroestradiol, 16-alpha-gitoxin, 16-epiestriol, 17-alpha dihydroequilenin, 17-alpha estradiol, 17-beta estradiol, 17-hydroxy progesterone, 1-alpha-hydroxyvitamin D2, 1-dodecpyrrolidinone, 20-epi-1,25 dihydroxyvitamin D3, 22-oxacalcitriol, 2CW, 2′-nor-cGMP, 3-isobutyl GABA, 5-ethynyluracil, 6-FUDCA, 7-methoxytacrine, abamectin, abanoquil, abcizimab (commercially available as REOPRO® from Eli Lilly and Company), abecarnil, abiraterone, ablukast, ablukast sodium, acadesine, acamprosate, acarbose, acebutolol, acecamide hydrochloride, aceclidine, aceclofenae, acedapsone, aceglutamide aluminum, acemannan, acetaminophen, acetazolamide, acetohexamide, acetohydroxamic acid, acetomepregenol, acetophenazine maleate, acetosulfone sodium, acetylcholine chloride, acetylcysteine, acetyl-L-carnitine, acetylmethadol, acifran, acipimox, acitemate, acitretin, acivicin, aclarubicin, aclatonium, acodazole hydrochloride, aconiazide, acrisorcin, acrivastine, acronine, actisomide, actodigin, acyclovir, acylfulvene, adafenoxate, adalimumab (commercially available as HUMIRA® from Abbott Laboratories), adapalene, adapalene, adatanserin, adatanserin hydrochloride, adecypenol, adecypenol, adefovir, adelmidrol, ademetionine, adenosine, adinazolam, adipheinine hydrochloride, adiposin, adozelesin, adrafinil, adrenalone, airbutamine, alacepril, alamecin, alanine, alaproclate, alaptide, albendazole, albolabrin, albuterol (commercially available as VENTOLIN® from GlaxoSmithKline), albutoin, alclofenae, alclometasone dipropionate, aluminum chlorhydroxyallantoinate (commercially available as ALCOLOXA® from TR1-K Industries, Inc.), aldecalmycin, aldesleukin, aldioxa, alendronate sodium (commercially available as FOSAMAX® from Merck), alendronic acid, alentemol, alentemol hydrobromide, aletamine hydrochloride, aleuronium chloride, alexidine, alfacalcidol, alfentanil hydrochloride, alfuzosin, algestone acetonide, alglucerase, aliflurane, alinastine, alipamide, allantoin, allobarbital, allopurinol, a tachy-kinins (TK) antagonist, alonimid, alosetron, alosetron hydrochloride, alovudine, alpertine, alpha amylase, alpha idosone, alpidem, alprazolam (commercially available as XANAX® from Pfizer, Inc.), alprenolol hydrochloride, alprenoxime hydrochloride, alprostadil, alrestatin sodium, altanserin tartrate, alteplase, althiazide, altretamine, altromycin B, alverinc citrate, alvircept sudotox, amadinone acetate, amantadine hydrochloride, ambamustine, ambomycin, ambruticin, ambuphylline, ambuside, amcinafal, amcinonide, amdinocillin, amdinocillin pivoxil, amedalin hydrochloride, amelometasone, ameltolide, amesergide, ametantrone acetate, amezinium metilsulfate, amfebutamone, amfenac sodium, amflutizole, amicycline, amidephrine mesylate, amidox, amifloxacin, amifostine, amikacin, amiloride hydrochloride, aminacrine hydrochloride, aminobenzoate potassium, aminobenzoate sodium, aminocaproic acid, aminoglutethimide, aminohippurate sodium, aminolevulinic acid, aminophylline, a minorex, aminosalicylate sodium, aminosalicylic acid, amiodarone, amiprilose hydrochloride, amiquinsin hydrochloride, amisulpride, amitraz, amitriptyline hydrochloride, amlexanox, amlodipine, amobarbital sodium, amodiaquine, amodiaquine hydrochloride, amorolfine, amoxapine, amoxicillin, amphecloral, amphetamine sulfate, amphomycin, amphotericin B, ampicillin, ampiroxicam, ampyzine sulfate, amquinate, aminone, aminone, amrubicin, amsacrine, amythiamicin, anagestone acetate, anagrelide, anakinra, ananain, anaritide, anaritide acetate, anastrozole (commercially available as ARIMIDEX® from AstraZeneca), anazolene sodium, ancrod, andrographolide, androstenedione, angiogenesis inhibitors, angiotensin amide, anidoxime, anileridine, anilopam hydrochloride, aniracetam, anirolac, anisotropine methylbromide, anistreplase, anitrazafen, anordrin, antagonist D, antagonist G, antarelix, antazoline phosphate, anthelmycin, anthralin, anthramycin, antiandrogen, antihemophilic factor (commercially available as XYNTHA® from Pfizer, Inc.), acedapsone, felbamate, antiestrogen, antineoplaston, antipyrine, antisense oligonucleotides, apadoline, apafant, apalcillin sodium, apaxifylline, apazone, aphidicolin glycinate, apixifylline, apomorphine hydrochloride, apraclonidine, apraclonidine hydrochloride, apramycin, aprindine, aprindine hydrochloride, aprosulate sodium, aprotinin, aptazapine maleate, aptiganel, apurinic acid, apurinic acid, aranidipine, aranotin, arbaprostil, arbekicin, 1-methyl-2-((phenylthio) methyl)-3-carbethoxy-4-((dimethylamino) methyl)-5-hydroxy-6-bromindole (commercially available as ARBIDOL® from Masterlek), arbutamine hydrochloride, arclofenin, ardeparin sodium, (2R,4R)-1-[(2S)-5-(diaminomethylideneamino)-2-[[(3R)-3-methyl-1,2,3,4-tetrahydroquinolin-8-yl]sulfonylamino]pentanoyl]-4-methyl-piperidine-2-carboxylic acid (commercially available as ARGATROBAN® from GlaxoSmithKline), arginine, argipressin tannate, arildone, aripiprazol, arotinolol, arpinocid, arteflene, artilide fumarate, asimadoline, aspalatone, asparaginase, aspartic acid, aspartocin, asperfuran, aspirin, aspoxicillin, asprelin, astemizole, astromicin sulfate, asulacrine, atamestane, atenolol, atevirdine, atipamezole, atiprosin maleate, atolide, atorvastatin (commercially available as LIPITOR® from Pfizer, Inc.), atosiban, atovaquone, atpenin B, atracurium besylate, atrimustine, atrinositol, atropine, auranofin, aureobasidin A, aurothioglucose, avilamycin, avoparcin, pyridine, nizatidine (commercially available as AXID® from GlaxoSmithKline), axinastatin 1, axinastatin 2, axinastatin 3, azabon, azacitidinie, azaclorzine hydrochloride, azaconazole, azadirachtine, azalanstat dihydrochloride, azaloxan fumarate, azanator maleate, azanidazole, azaperone, azaribine, azaserine, azasetron, azatadine maleate, azathioprine, azathioprine sodium, azatoxin, azatyrosine, azelaic acid, azelastine, azelnidipine, azepindole, azetepa, azimilide, azithromycin, azlocillin, azolimine, azosemide, azotomycin, aztreonam, azumolene sodium, bacampicillin hydrochloride, baccatin III, bacitracin, baclofen, bacoside A, bacoside B, bactobolamine, balanol, balazipone, balhimycin, balofloxacin, balsalazide, bambermycins, bambuterol, bamethan sulfate, bamifylline hydrochloride, bamidazole, baohuoside 1, barmastine, barnidipine, basifungin, batanopride hydrochloride, batebulast, batelapine maleate, batimastat, beauvericin, becanthone hydrochloride, becaplermin, becliconazole, beclomethasone dipropionate, befloxatone, beinserazide, belfosdil, belladonna, beloxamide, bemesetron, bemitradine, bemoradan, benapryzine hydrochloride, benazepril hydrochloride, benazeprilat, bendacalol mesylate, bendazac, bendroflumethiazide, benflumetol, benidipine, benorterone, benoxaprofen, benoxaprofen, benoxinate hydrochloride, benperidol, bentazepam, bentiromide, benurestat, benzbromarone, benzethonium chloride, benzetimide hydrochloride, benzilonium bromide, benzindopyrine hydrochloride, benzisoxazole, benzocaine, benzochlorins, benzoctamine hydrochloride, benzodepa, benzoidazoxan, benzonatate, benzoyl peroxide, benzoylpas calcium, benzoylstaurosporine, benzquinamide, benzthiazide, benztropine, benztropine mesylate, benzydamine hydrochloride, benzylpenicilloyl polylysine, bepridil, bepridil hydrochloride, beractant, beraprost, berefrine, berlafenone, bertosamil, berythromycin, besipirdine, beta-alethine, betaclamycin B, betamethasone, betamipron, betaxolol, betaxolol hydrochloride, bethanechol chloride, bethanidine sulfate, betulinic acid, bevacizumab (commercially available as AVASTIN® available from Genenetech), bevantolol, bevantolol hydrochloride, bezafibrate, bFGF inhibitor, bialamicol hydrochloride, biapenem, bicalutamide, bicifadine hydrochloride, biclodil hydrochloride, bidisomide, bifemelane, bifonazole, bimakalim, bimithil, bindarit, biniramycin, binospirone, bioxalomycin alpha2, bipenamol hydrochloride, biperiden, biphenamine hydrochloride, biriperone, bisantrene, bisaramil, bisaziridinylspermine, bis-benzimidazole A, bis-benzimidazole B, bisnafide, bisobrin lactate, bisoprolol, bispyrithione magsulfex, bistramide D, bistramide K, bistratene A, bithionolate sodium, bitolterol besylate, bivalirudin, bizelesin, bleomycin sulfate, bolandiol dipropionate, bolasterone, boldenone undecylenate, boldine, bolenol, bolmantalate, bopindolol, bosentan, boxidine, brefeldin, breflate, brequinar sodium, bretazenil, bretylium bosylate, brifentanil hydrochloride, brimonidine, brinolase, brocresine, brocrinat, brofoxine, bromadoline maleate, bromazepam, bromchlorenone, bromelains, bromfenac, brominidione, bromocriptine, bromodiphenhydramine hydrochloride, bromoxamide, bromperidol, bromperidol decanoate, brompheniramine baleate, broperamole, bropirimine, brotizolam, bucamide maleate, bucindolol, buclizine hydrochloride, bucromarone, budesonide (commercially available as RHINOCORT® and ENTOCORT® from AstraZeneca), budipine, budotitane, buformin, bumetanide, bunaprolast, bunazosin, bunolol hydrochloride, bupicomide, bupivacaine hydrochloride, buprenorphine hydrochloride, bupropion hydrochloride, buramate, buserelin acetate, buspirone hydrochloride, busulfan, butabarbital, butacetin, butaclamol hydrochloride, butalbital, butamben, butamirate citrate, butaperazine, butaprost, butedronate tetrasodium, butenafine, buterizine, buthionine sulfoximine, butikacin, butilfenin, butirosin sulfate, butixirate, butixocort propionate, butoconazole nitrate, butonate, butopamine, butoprozine hydrochloride, butorphanol, butoxamine hydrochloride, butriptyline hydrochloride, cactinomycin, cadexomer iodine, caffeine, calanolide A, calcifediol, calcipotriene, calcipotriol, calcitonin, calcitriol, calcium undecylenate, calphostin C, calusterone, cambendazole, camonagrel, camptothecin derivatives, canarypox IL-2, candesartan, candicidin, candoxatril, candoxatrilat, caniglibose, canrenoate potassium, canrenone, capecitabine, capobenate sodium, capobenic acid, capreomycin sulfate, capromab, capsaicin, captopril, capuride, caracemide, carbachol, carbadox, carbamazepine, carbamide peroxide, carbantel lauryl sulfate, carbaspirin calcium, carbazeran, carbazomycin C, carbenicillin potassium, carbenoxolone sodium, carbetimer, carbetocin, carbidopa, carbidopa-levodopa, carbinoxamine maleate, carbiphene hydrochloride, carbocloral, carbocysteine, carbol-fuchsin, carboplatin, carboprost, carbovir, carboxamide-amino-triazole, carboxyamidotriazole, carboxymethylated beta-1,3-glucan, carbuterol hydrochloride, CaRest M3, carfentanil citrate, carisoprodol, carmantadine, carmustine, CARN 700, camidazole, caroxazone, carperitide, carphenazine maleate, carprofen, carsatrin succinate, cartazolate, carteolol, carteolol hydrochloride, cartilage derived inhibitor, carubicin hydrochloride, carumonam sodium, carvedilol, carvotroline, carvotroline hydrochloride, carzelesin, casein kinase inhibitors (ICOS), castanospermine, caurumonam, cebaracetam, cecropin B, cedefingol, cefaclor, cefadroxil, cefamandole, cefaparole, cefatrizine, cefazaflur sodium, cefazolin, cefbuperazone, cefcapene pivoxil, cefdaloxime pentexil tosilate, cefdinir, cefditoren pivoxil, cefepime, cefetamet, cefetecol, cefixime, cefluprenam, cefinenoxime hydrochloride, cefinetazole, cefminlox, cefodizime, cefonicid sodium, cefoperazone sodium, ceforamide, cefoselis, cefotaxime sodium, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftibuten, ceftizoxime sodium, ceftriaxone, cefuroxime, celastrol, celikalim, celiprolol, cepacidiine A, cephacetrile sodium, cephalexin, cephaloglycin, cephaloridine, cephalothin sodium, cephapirin sodium, cephradine, cericlamine, cerivastatin, ceronapril, certoparin sodium, ceruletide, cetaben sodium, cetalkonium chloride, cetamolol hydrochloride, cetiedil, cetirizine, cetophenicol, cetraxate hydrochloride, cetrorelix, cetuximab (commercially available as ERBITUX® from Eli Lilly and Company), cetylpyridinium chloride, chenodiol, chlophedianol hydrochloride, chloral betaine, chlorambucil, chloramphenicol, chlordantoin, chlordiazepoxide, chlorhexidine gluconate, chlorins, chlormadinone acetate, chloroorienticin A, chloroprocaine hydrochloride, chloropropamide, chloroquine, chloroquinoxaline sulfonamide, chlorothiazide, chlorotrianisene, chloroxine, chloroxylenol, chlorphenesin carbamate, chlorpheniramine maleate, chlorpromazine, chlorpropamide, chlorprothixene, chlortetracycline bisulfate, chlorthalidone, chlorzoxazone, cholestyramine resin, chromonar hydrochloride, cibenzoline, cicaprost, ciclafrine hydrochloride, ciclazindol, ciclesonide, cicletanine, ciclopirox, cicloprofen, cicloprolol, cidofovir, cidoxepin hydrochloride, cifenline, ciglitazone, ciladopa hydrochloride, cilansetron, cilastatin sodium, cilazapril, cilnidipine, cilobamine mesylate, cilobradine, cilofungin, cilostazol, cimaterol, cimetidine, cimetropium bromide, cinalukast, cinanserin hydrochloride, cinepazet maleate, cinflumide, cingestol, cinitapride, cinnamedrine, cinnarizine, cinolazepam, cinoxacin, cinperene, cinromide, cintazone, cintriamide, cioteronel, cipamfylline, ciprefadol succinate, ciprocinonide, ciprofibrate, ciprofloxacin, ciprostene, ciramadol, cirolemycin, cisapride, cisatracurium besilate, cisconazole, cisplatin, cis-porphyrin, cistinexine, citalopram, citenamide, citicoline, citreamicin alpha, cladribine, clamoxyquin hydrochloride, clarithromycin, clausenamide, clavulanate potassium, clazolam, clazolimine, clebopride, clemastine, Clentiazem maleate, clidinium bromide, clinafloxacin, clindamycin, clioquinol, clioxamide, cliprofen, clobazam, clobetasol propionate, clobetasone butyrate, clocortolone acetate, clodanolene, clodazon hydrochloride, clodronic acid, clof azimine, clofibrate, clofilium phosphate, clogestone acetate, clomacran phosphate, clomegestone acetate, clometherone, clomethiazole, clomifene analogues, clominorex, clomiphene, clomipramine hydrochloride, clonazepam, clonidine, clonitrate, clonixeril, clonixin, clopamide, clopenthixol, cloperidone hydrochloride, clopidogrel (commercially available as PLAVIX® from Bristol-Myers Squibb and Sanofi Pharmaceuticals), clopimozide, clopipazan mesylate, clopirac, cloprednol, cloprostenol sodium, clorazepate dipotassium, clorethate, clorexolone, cloroperone hydrochloride, clorprenaline hydrochloride, clorsulon, clortermine hydrochloride, closantel, closiramine aceturate, clothiapine, clothixamide maleate cloticasone propionate, clotrimazole, cloxacillin benzathine, cloxyquin, clozapine, cocaine, coccidioidin, codeine, codoxime, colchicine, colestimide, colestipol hydrochloride, colestolone, colforsin, colfosceril palmitate, colistimethate sodium, colistin sulfate, collismycin A, collismycin B, colterol mesylate, combretastatin A4, combretastatin analogue, complestatin, conagenin, conorphone hydrochloride, contignasterol, contortrostatin, cormethasone acetate, corticorelin ovine triflutate, corticotropin, cortisone acetate, cortivazol, cortodoxone, cosalane, costatolide, cosyntropin, cotinine, warfarin (commercially available as COUMADIN® from Bristol-Myers Squibb), coumermycin, crambescidin 816, crilvastatin, crisnatol, cromitrile sodium, cromolyn sodium, crotamiton, cryptophycin 8, cucumariosid, cuprimyxin, curacin A, curdlan sulfate, zinc hyaluran (commercially available as CURIOSIN® from Gedeon Richter), cyclacillin, cyclazocine, cyclazosin, cyclic HPMPC, cyclindole, cycliramine maleate, cyclizine, cyclobendazole, cyclobenzaprine, cyclobut A, cyclobut G, cyclocapron, cycloguanil pamoate, cycloheximide, cyclopentanthraquinones, cyclopenthiazide, cyclopentolate hydrochloride, cyclophenazine hydrochloride, cyclophosphamide, cycloplatam, cyclopropane, cycloserine, cyclosin, cyclosporine, cyclothialidine, cyclothiazide, cyclothiazomycin, cyheptamide, cypemycin, cypenamine hydrochloride, cyprazepam, cyproheptadine hydrochloride, cyprolidol hydrochloride, cyproterone, cyproximide, cysteamine, cysteine hydrochloride, cystine, cytarabine, cytarabine hydrochloride, cytarabine ocfosfate, cytochalasin B, cytolytic factor, cytostatin, dacarbazine, dacliximab, dactimicin, dactinomycin, daidzein, daledalin tosylate, dalfopristin, dalteparin sodium, daltroban, dalvastatin, danaparoid, danazol, dantrolene, daphlnodorin A, dapiprazole, dapitant, dapoxetine hydrochloride, dapsone, daptomycin, darglitazone sodium, darifenacin, darlucin A, darodipine, darsidomine, darusentan, daunorubicin hydrochloride, dazadrol maleate, dazepinil hydrochloride, dazmegrel, dazopride fumarate, dazoxiben hydrochloride, debrisoquin sulfate, decitabine, deferiprone, deflazacort, dehydrocholic acid, dehydrodidemnin B, dehydroepiandrosterone, delapril, delapril hydrochloride, delavirdine mesylate, delequamine, delfaprazine, delmadinone acetate, delmopinol, delphinidin, demecarium bromide, demeclocycline, demecycline, demoxepam, denofungin, deoxypyridinoline, 2-propylpentanoic acid (commercially available as DEPAKOTE® from Abbott), deprodone, deprostil, depsidomycin, deramciclane, dermatan sulfate, desciclovir, descinolone acetonide, desflurane, desipramine hydrochloride, desirudin, deslanoside, deslorelin, desmopressin, desogestrel, desonide, desoximetasone, desoxoamiodarone, desoxycorticosterone acetate, detajmium bitartrate, deterenol hydrochloride, detirelix acetate, devazepide, dexamethasone, dexamisole, dexbrompheniramine maleate, dexchlorpheniramine maleate, dexclamol hydrochloride, dexetimide, dexfenfluramine hydrochloride, dexifosfamide, deximafen, dexivacaine, dexketoprofen, dexloxiglumide, dexmedetomidine, dexormaplatin, dexoxadrol hydrochloride, dexpanthenol, dexpemedolac, dexpropranolol hydrochloride, dexrazoxane, dexsotalol, dextrin 2-sulphate, dextroamphetamine, dextromethorphan, dextrorphan hydrochloride, dextrothyroxine sodium, dexverapamil, dezaguanine, dezinamide, dezocine, diacetolol hydrochloride, diamocaine cyclamate, diapamide, diatrizoate meglumine, diatrizoic acid, diaveridine, diazepam, diaziquone, diazoxide, dibenzepin hydrochloride, dibenzothiophene, dibucaine, dichliorvos, dichloralphenazone, dichlorphenamide, dicirenone, diclofenac sodium, dicloxacillin, dicranin, dicumarol, dicyclomine hydrochloride, didanosine, didemnin B, didox, dienestrol, dienogest, diethylcarbamazine citrate, diethylhomospermine, diethylnorspermine, diethylpropion hydrochloride, diethylstilbestrol, difenoximide hydrochloride, difenoxin, diflorasone diacetate, difloxacin hydrochloride, difluanine hydrochloride, diflucortolone, diflumidone sodium, diflunisal, difluprednate, diftalone, digitalis, digitoxin, digoxin, dihexyverine hydrochloride, dihydrexidine, dihydro-5-azacytidine, dihydrocodeine bitartrate, dihydroergotamine mesylate, hihydroestosterone, dihydrostreptomycin sulfate, dihydrotachysterol, dihydrotaxol, phenyloin (commercially available as DILANTIN® from Parke, Davis & Company), dilevalol hydrochloride, diltiazem hydrochloride, dimefadane, dimefline hydrochloride, dimenhydrinate, dimercaprol, dimethadione, dimethindene maleate, dimethisterone, dimethyl prostaglandin A1, dimethyl sulfoxide, dimethylhomospermine, dimiracetam, dimoxamine hydrochloride, dinoprost, dinoprostone, dioxadrol hydrochloride, dioxamycin, diphenhydramine citrate, diphenidol, diphenoxylate hydrochloride, diphenyl spiromustine, dipivefin hydrochloride, dipivefrin, dipliencyprone, diprafenone, dipropylnorspermine, dipyridamole, dipyrithione, dipyrone, dirithromycin, discodermolide, disobutamide, disofenin, disopyramide, disoxaril, disulfuram, ditekiren, divalproex sodium, dizocilpine maleate, dobutamine, docarpamine, docebenone, docetaxel, doconazole, docosanol, dofetilide, dolasetron, drotrecogin alfa (commercially available as XIGRIS® from Eli Lilly and Company), duloxetine hydrochloride (commercially available as CYMBALTA® from Eli Lilly and Company), ebastine, ebiratide, ebrotidine, ebselen, ecabapide, ecabet, ecadotril, ecdisteron, echicetin, echistatin, echothiophate iodide, eclanamine maleate, eclazolast, ecomustine, econazole, ecteinascidin 722, edaravone, edatrexate, edelfosine, edifolone acetate, edobacomab, edoxudine, edrecolomab, edrophonium chloride, edroxyprogesteone acetate, efegatran, eflornithine, efonidipine, egualcen, elantrine, eleatonin, elemene, eletriptan, elgodipine, eliprodil, elsamitrucin, eltenae, elucaine, emalkalim, emedastine, emetine hydrochloride, emiglitate, emilium tosylate, emitefur, emoctakin, enadoline hydrochloride, enalapril, enalaprilat, enalkiren, enazadrem, encyprate, endralazine mesylate, endrysone, enflurane, englitazone, enilconazole, enisoprost, enlimomab, enloplatin, enofelast, enolicam sodium, enoxacin, enoxacin, enoxaparin sodium, enoxaparin sodium, enoximone, enpiroline phosphate, enprofylline, enpromate, entacapone, enterostatin, enviradene, enviroxime, ephedrine, epicillin, epimestrol, epinephrine, epinephryl borate, epipropidine, epirizole, epirubicin, epitetracycline hydrochloride, epithiazide, epoetin alfa, epoetin beta, epoprostenol, epoprostenol sodium, epoxymexrenone, epristeride, eprosartan, eptastigmine, equilenin, equilin, erbulozole, erdosteine, ergoloid mesylates, ergonovine maleate, ergotamine tartrate, ersentilide, ersofermin, erythritol, erythrityl tetranitrate, erythromycin, esmolol hydrochloride, esomeprazole (commercially available as NEXIUM® from AstraZeneca), esorubicin hydrochloride, esproquin hydrochloride, estazolam, estradiol, estramustine, estramustine analogue, estrazinol hydrobromide, estriol, estrofurate, estrogen agonists, estrogen antagonists, estrogens, conjugated estrogens, esterified, estrone, estropipate, esuprone, etafedrine hydrochloride, etanidazole, etanterol, etarotene, etazolate hydrochloride, eterobarb, ethacizin, ethacrynate sodium, ethacrynic acid, ethambutol hydrochloride, ethamivan, ethanolamine oleate, ethehlorvynol, ether, ethinyl estradiol, ethiodized oil, ethionamide, ethonam nitrate, ethopropazine hydrochloride, ethosuximide, ethotoin, ethoxazene hydrochloride, ethybenztropine, ethyl chloride, ethyl dibunate, ethylestrenol, ethyndiol, ethynerone, ethynodiol diacetate, etibendazole, etidocaine, etidronate disodium, etidronic acid, etifenin, etintidine hydrochloride, etizolam, etodolac, etofenamate, etoformin hydrochloride, etomidate, etonogestrel, etoperidone hydrochloride, etoposide, etoprine, etoxadrol hydrochloride, etozolin, etrabamine, etretinate, etryptamine acetate, eucatropine hydrochloride, eugenol, euprocin hydrochloride, eveminomicin, exametazine, examorelin, exaprolol hydrochloride, exemestane, exetimibe (commercially available as ZETIA® from Merck), fadrozole, faeriefungin, famciclovir, famotidine (commercially available as PEPCID® from Merck), fampridine, fantof arone, fantridone hydrochloride, faropenem, fasidotril, fasudil, fazarabine, fedotozine, felbamate, felbinac, felodipine, felypressin, fenalamide, fenamole, fenbendazole, fenbufen, fencibutirol, fenclofenac, fenclonine, fenclorac, fendosal, fenestrel, fenethylline hydrochloride, fenfluramine hydrochloride, fengabine, fenimide, fenisorex, fenmetozole hydrochloride, fenmetramide, fenobam, fenoctimine sulfate, fenofibrate, fenoldopam, fenoprofen, fenoterol, fenpipalone, fenprinast hydrochloride, fenprostalene, fenquizone, fenretinide, fenspiride, fentanyl citrate, fentiazac, fenticlor, fenticonazole, fenyripol hydrochloride, fepradinol, ferpifosate sodium, ferristene, ferrixan, ferrous sulfate, ferumoxides, ferumoxsil, fetoxylate hydrochloride, fexofenadine, fezolamine fumarate, fiacitabine, fialuridine, fibrinogen 1125, filgrastim, filipin, finasteride (commercially available as PROPECIA® from Merck), flavodilol maleate, flavopiridol, flavoxate hydrochloride, flazalone, flecamide, flerobuterol, fleroxacin, flesinoxan, flestolol sulfate, fletazepam, flezelastine, flobufen, floctafenine, flomoxef, flordipine, florfenicol, florifenine, flosatidil, flosequinan, floxacillin, floxuridine, fluasterone, fluazacort, flubanilate hydrochloride, flubendazole, flucindole, flucloronide, fluconazole, flucytosine, fludalanine, fludarabine phosphate, fludazonium chloride, fludeoxyglucose F 18, fludorex, fludrocortisone acetate, flufenamic acid, flufenisal, flumazenil, flumecinol, flumequine, flumeridone, flumethasone, flumetramide, flumezapine, fluminorex, flumizole, flumoxonide, flunarizine, flunidazole, flunisolide, flunitrazepam, flunixin, fluocalcitriol, fluocinolone acetonide, fluocinonide, fluocortin butyl, fluocortolone, fluorescein, fluorodaunorunicin hydrochloride, fluorodopa F 18, fluoroformylone, fluoroquinolones, fluorometholone, fluorouracil, fluotracen hydrochloride, fluoxetine, fluoxymesterone, fluparoxan, fluperamide, fluperolone acetate, fluphenazine decanoate, flupirtine, fluprednisolone, fluproquazone, fluprostenol sodium, fluquazone, fluradoline hydrochloride, flurandrenolide, flurazepam hydrochloride, flurbiprofen, fluretofen, flurithromycin, fluorocitabine, fluorof amide, fluorogestone acetate, fluorothyl, fluoroxene, fluspiperone, fluspirilene, fluticasone propionate (commercially available as ADVAIR® from GlaxoSmithKline), fluticasone furoate, flutrimazole, flutroline, fluvastatin, fluvastatin sodium, fluvoxamine, fluzinamide, folic acid, follicle regulatory protein, folliculostatin, fomepizole, fonazine mesylate, forasartan, forfenimex, forfenirmex, formestane, formocortal, formoterol, fosarilate, fosazepam, foscarnet sodium, fosfomycin, fosfonet sodium, fosinopril, fosinoprilat, fosphenyloin, fosquidone, fostedil, fostriecin, fotemustine, fuchsin, basic, fumoxicillin, fungimycin, furaprofen, furazolidone, furazolium chloride, furegrelate sodium, furobufen, furodazole, furosemide, fusidate sodium, fusidic acid, gabapentin, gadobenate dimeglumine, gadobenic acid, gadobutrol, gadodiamide, gadolinium texaphyrin, gadopentetate dimegiumine, gadoteric acid, gadoteridol, gadoversetamide, galantamine, galdansetron, galdansetron hydrochloride, gallamine triethiodide, gallium nitrate, gallopamil, galocitabine, gamfexine, gamolenic acid, ganciclovir, ganirelix, ganirelix acetate, gelatinase inhibitors, gemcadiol, gemcitabine (commercially available as GEMZAR® from Eli Lilly and Company), gemeprost, gemfibrozil, gentamicin sulfate, gentian violet, gepirone, gestaclone, gestodene, gestonorone caproate, gestrinone, gevotroline hydrochloride, girisopam, glaspimod, glaucocalyxin A, glemanserin, gliamilide, glibornuride, glicetanile sodium, gliflumide, glimepiride, glipizide, gloximonam, glucagon, glutapyrone, glutathione inhibitors, glutethimide, glyburide, glycopine, glycopril, glycopyrrolate, glyhexamide, glymidine sodium, glyoctamide, glyparamide, colloidal gold Au 198, gonadoctrinins, gonadorelin, gonadotropins, goserelin, gramicidin, granisetron, grepafloxacin, griseofulvin, guaiapate, guaithylline, guanabenz, guanabenz acetate, guanadrel sulfate, guancydine, guanethidine monosulfate, guanfacine hydrochloride, guanisoquin sulfate, guanoclor sulfate, guanoctine hydrochloride, guanoxabenz, guanoxan sulfate, guanoxyfen sulfate, gusperimus trihydrochloride, halazepam, halcinonide, halichondrin B, halobetasol propionate, halof antrine, halof antrine hydrochloride, halofenate, halofuginone hydrobromide, halomon, galopemide, galoperidol, halopredone, haloprogesterone, haloprogin, halothane, halquinols, hamycin, han menopausal gonadotropins, hatomamicin, hatomarubigin A, hatomarubigin B, hatomarubigin C, hatomarubigin D, heparin sodium, hepsulfam, heregulin, hetacillin, heteronium bromide, hexachlorophene: hydrogen peroxide, hexafluorenium bromide, hexamethylene bisacetamide, hexedine, hexobendine, hexoprenaline sulfate, hexylresorcinol, histamine phosphate, histidine, histoplasmin, histrelin, homatropine hydrobromide, hoquizil hydrochloride, human chorionic gonadotropin, hycanthone, hydralazine hydrochloride, hydralazine polistirex, hydrochlorothiazide, hydrocodone bitartrate, hydrocortisone, hydroflumethiazide, hydromorphone hydrochloride, hydroxyamphetamine hydrobromide, hydroxychloroquine sulfate, hydroxyphenamate, hydroxyprogesterone caproate, hydroxyurca, hydroxyzine hydrochloride, hymecromone, hyoscyamine, hypericin, ibafloxacin, ibandronic acid, ibogaine, ibopamine, ibudilast, ibufenac, ibuprofen, ibutilide fumarate, icatibant acetate, ichthammol, icotidine, idarubicin, idoxifene, idoxuridine, idramantone, iemefloxacin, iesopitron, ifetroban, ifosfamide, ilepeimide, illimaquinone, ilmofosine, ilomastat, ilonidap, iloperidone, iloprost, imafen hydrochloride, imazodan hydrochloride, imidapril, imidazenil, imidazoacridones, imidecyl iodine, imidocarb hydrochloride, imidoline hydrochloride, imidurea, imiloxan hydrochloride, imipenem, imipramine hydrochloride, imiquimod, immunostimulant peptides, impromidine hydrochloride, indacrinone, indapamide, indecamide hydrochloride, indeloxazine hydrochloride, indigotindisulfonate sodium, indinavir, indocyanine green, indolapril hydrochloride, indolidan, indometacin, indomethacin sodium, indoprofen, indoramin, indorenate hydrochloride, indoxole, indriline hydrochloride, infliximab (commercially available as REMICADE® from Janssen Biotech, Inc.), inocoterone, inogatran, inolimomab, inositol niacinate, insulin, insulin glargine (commercially available as LANTUS® from Sanofi-Aventis), interferons, interferon beta-1a (commercially available as AVONEX® from BIOGEN), interleukins, intrazole, intriptyline hydrochloride, iobenguane, iobenzamic acid, iobitridol, iocarmate meglumine, iocarmic acid, iocetamic acid, iodamide, iodine, iodipamide meglumine, iodixanol, iodoamiloride, iodoantipyrine I 131, iodocholesterol I 131, iododoxorubicin, iodohippurate sodium I 131, iodopyracet I 125, iodoquinol, iodoxamate meglumine, iodoxamie acid, ioglicic acid, iofetamine hydrochloride I 123, iofratol, ioglucol, ioglucomide, ioglycamic acid, iogulamide, iohexyl, iomeprol, iomethin I 125, iopamidol, iopanoic acid, iopentol, iophendylate, ioprocemic acid, iopromide, iopronic acid, iopydol, iopydone, iopyrol, iosefamic acid, ioseric acid, iosulamide meglumine, iosumetic acid, iotasul, iotetric acid, iothalamate sodium, iothalamic acid, iotriside, iotrolan, iotroxic acid, iotyrosine I 131, ioversol, ioxagiate sodium, ioxaglate meglumine, ioxaglic acid, ioxilan, ioxotrizoic acid, ipazilide, ipenoxazone, ipidacrine, ipodate calcium, ipomeanol, 4-, ipratropium bromide, ipriflavone, iprindole, iprofenin, ipronidazole, iproplatin, iproxamine hydrochloride, ipsapirone, irbesartan, irinotecan, irloxacin, iroplact, irsogladine, irtemazole, isalsteine, isamoxole, isbogrel, isepamicin, isobengazole, isobutamben, isocarboxazid, isoconazole, isoetharine, isofloxythepin, isoflupredone acetate, isoflurane, isofluorophate, isohomohalicondrin B, isoleucine, isomazole hydrochloride, isomylamine hydrochloride, isoniazid, isopropamide iodide, isopropyl alcohol, isopropyl unoprostone, isoproterenol hydrochloride, isosorbide, isosorbide mononitrate, isotiquimide, isotretinoin, isoxepac, isoxicam, isoxsuprine hydrochloride, isradipine, itameline, itasetron, itazigrel, itopride, itraconazole, ivermectin, jasplakinolide, josamycin, kahalalide F, kalafungin, kanamycin sulfate, ketamine hydrochloride, ketanserin, ketazocine, ketazolam, kethoxal, ketipramine fumarate, ketoconazole, ketoprofen, ketorfanol, ketorolac, ketotifen fumarate, kitasamycin, labetalol hydrochloride, lacidipine, lacidipine, lactitol, lactivicin, laennec, lafutidine, lamellarin-n triacetate, lamifiban, lamivudine, lamotrigine, lanoconazole, LANOXIN® (digoxin, available from GlaxoSmithKline), lanperisone, lanreotide, lansoprazole (commercially available as PREVAID® from Takeda Pharmaceuticals, Inc.), latanoprost, lateritin, laurocapram, lauryl isoquinolinium bromide, lavoltidine succinate, lazabemide, lecimibide, leinamycin, lemildipine, leminoprazole, lenercept, leniquinsin, lenograstim, lenperone, lentinan sulfate, leptin, leptolstatin, lercanidipine, lergotrile, lerisetron, letimide hydrochloride, letrazuril, letrozole, leucine, leucomyzin, leuprolide acetate, leuprolide, leuprorelin, levamfetamine succinate, levamisole, levdobutamine lactobionate, levcromakalim, levetiracetam, levobetaxolol, levobunolol, levobupivacaine, levocabastine, levocarnitine, levodopa, levodropropizine, levofloxacin (commercially available as LEVAQUIN® from Jessen Pharmaceuticals, Inc.), levofuraltadone, levoleucovorin calcium, levomethadyl acetate, levomethadyl acetate hydrochloride, levomoprolol, levonantradol hydrochloride, levonordefrin, levonorgestrel, levopropoxyphene napsylate, levopropylcillin potassium, levormeloxifene, levorphanol tartrate, levosimendan, levosulpiride, levothyroxine sodium, levoxadrol hydrochloride, lexipafant, lexithromycin, liarozole, libenzapril, lidamidine hydrochloride, lidocaine, lidofenin, lidoflazine, lifarizine, lifibrate, lifibrol, linarotene, lincomycin, linear polyamine analogue, linogliride, linopirdine, linotroban, linsidomine, lintitript, lintopride, liothyronine I 125, liothyronine sodium, liotrix, lirexapride, lisinopril, lissoclinamide 7, lixazinone sulfate, lobaplatin, lobenzarit sodium, lobucavir, lodelaben, lodoxamide, lofemizole hydrochloride, lofentanil oxalate, lofepramine hydrochloride, lofexidine hydrochloride, lombricine, lomefloxacin, lomerizine, lometraline hydrochloride, lometrexol, lomitapide, lomofungin, lornoxicam, lomustine, lonapalene, lonazolac, lonidamine, loperamide hydrochloride, loracarbef, lorajmine hydrochloride, loratadine, lorazepam, lorbamate, lorcamide hydrochloride, loreclezole, lorglumide, lormetazepam, lornoxicam, lornoxicam, lortalamine, lorzafone, losartan (commercially available as COZAAR® from Merck), losigamone, losoxantrone, losulazine hydrochloride, loteprednol, lovastatin, loviride, loxapine, loxoribine, lubeluzole, lucanthone hydrochloride, lufironil, lurosetron mesylate, lurtotecan, luteinizing hormone, lutetium, lutrelin acetate, luzindole, lyapolate sodium, lycetamine, lydicamycin, lydimycin, lynestrenol, lypressin, lysine, lysofylline, lysostaphin, lytic peptides, maduramicin, mafenide, magainin 2 amide, magnesium salicylate, magnesium sulfate, magnolol, maitansine, malethamer, mallotochromene, mallotojaponin, malotilate, mangafodipir, manidipine, maniwamycin A, mannitol, mannostatin A, manumycin E, manumycin F, MAPK/ERK kinase (MEK) inhibitors, mapinastine, maprotiline, marimastat, masoprocol, maspin, massetolide, matrilysin inhibitors, maytansine, mazapertine succiniate, mazindol, mebendazole, mebeverine hydrochloride, mebrofenin, mebutamate, mecamylamine hydrochloride, mechlorethamine hydrochloride, meclocycline, meclofenamate sodium, mecloqualone, meclorisone dibutyrate, medazepam hydrochloride, medorinone, medrogestone, medroxalol, medroxyprogesterone (commercially available as DEPO-PROVERA® from Pfizer, Inc.), medrysone, meelizine hydrochloride, mefenamic acid, mefenidil, mefenorex hydrochloride, mefexamide, mefloquine hydrochloride, mefruside, megalomicin potassium phosphate, megestrol acetate, meglumine, meglutol, melengestrol acetate, melitracen hydrochloride, melphalan, memotine hydrochloride, menabitan hydrochloride, menoctone, menogaril, menotropins, meobentine sulfate, mepartricin, mepenzolate bromide, meperidine hydrochloride, mephentermine sulfate, mephenyloin, mephobarbital, mepivacaine hydrochloride, meprobamate, meptazinol hydrochloride, mequidox, meralein sodium, merbarone, mercaptopurine, mercufenol chloride, mercury, meropenem, mesalamine, meseclazone, mesoridazine, mesterolone, mestranol, mesuprine hydrochloride, metalol hydrochloride, metaproterenol polistirex, metaraminol, bitartrate, metaxalone, meteneprost, meterelin, metformin, methacholine chloride, methacycline, methadone hydrochloride, methadyl acetate, methalthiazide, methamphetamine hydrochloride, methaqualone, methazolamide, methdilazine, methenamine, methenolone acetate, methetoin, methicillin sodium, methimazole, methioninase, methionine, methisazone, methixene hydrochloride, methocarbamol, methohexital sodium, methopholine, methotrexate, methotrimeprazine, methoxatone, methoxyflurane, methsuximide, methyclothiazide, methyl 10 palmoxirate, methylatropine nitrate, methylbenzethonium chloride, methyldopa, methyldopate hydrochloride, methylene blue, methylergonovine maleate, methylhistamine, R-alpha, methylinosine monophosphate, methylphenidate hydrochloride, methylprednisolone, methyltestosterone, methynodiol diacelate, methysergide, methysergide maleate, metiamide, metiapine, metioprim, metipamide, metipranolol, metizoline hydrochloride, metkephamid acetate, metoclopramide, metocurine iodide, metogest, metolazone, metopimazine, metoprine, metoprolol, metoquizine, metrifonate, metrizamide, metrizoate sodium, metronidazole, meturedepa, metyrapone, metyrosine, mexiletine hydrochloride, mexrenoate potassium, mezlocillin, mfonelic acid, mianserin hydrochloride, mibefradil, mibefradil dihydrochloride, mibolerone, michellamine B, miconazole, microcolin A, midaflur, midazolam hydrochloride, midodrine, mifepristone, mifobate, miglitol, milacemide, milameline, mildronate, milenperone, milipertine, milnacipran, milrinone, miltefosine, mimbane hydrochloride, minaprine, minaxolone, minocromil, minocycline, minoxidil, mioflazine hydrochloride, miokamycin, mipragoside, mirfentanil, mirimostim, mirincamycin hydrochloride, mirisetron maleate, mirtazapine, mismatched double stranded RNA, misonidazole, misoprostol, mitindomide, mitocarcin, mitocromin, mitogillin, mitoguazone, mitolactol, mitomalcin, mitomycin, mitonafide, mitosper, mitotane, mitoxantrone, mivacurium chloride, mivazerol, mixanpril, mixidine, mizolastine, mizoribine, moclobemide, modafinil, modaline sulfate, modecamide, moexipril, mof arotene, mofegiline hydrochloride, mofezolac, molgramostim, molinazone, molindone hydrochloride, molsidomine, mometasone, monatepil maleate, monensin, monoctanoin, montelukast sodium (commercially available as SINGULAIR® available from Merck), montirelin, mopidamol, moracizine, morantel tartrate, moricizine, morniflumate, morphine, morphine sulfate, morrhuate sodium, mosapramine, mosapride, motilide, motretinide, moxalactam disodium, moxazocine, moxiraprine, moxnidazole, moxonidine, mumps skin test antigen, mustard anticancer agent, muzolimine, mycaperoxide B, mycophenolic acid, myriaporone, nabazenil, nabilone, nabitan hydrochloride, naboctate hydrochloride, nabumetone, n-acetyldinaline, nadide, nadifloxacin, nadolol, nadroparin calcium, nafadotride, nafamostat, nafarelin, nafcillin sodium, nafenopin, nafimidone hydrochloride, naflocort, nafomine malate, nafoxidine hydrochloride, nafronyl oxalate, naftifine hydrochloride, naftopidil, naglivan, nagrestip, nalbuphine hydrochloride, nalidixate sodium, nalidixic acid, nalmefene, nalmexone hydrochloride, naloxone/pentazocine, naltrexone, namoxyrate, nandrolone phenpropionate, nantradol hydrochloride, napactadine hydrochloride, napadisilate, napamezole hydrochloride, napaviin, naphazoline hydrochloride, naphterpin, naproxen, naproxol, napsagatran, naranol hydrochloride, narasin, naratriptan, nartograstim, nasaruplase, natamycin, nateplase, naxagolide hydrochloride, nebivolol, nebramycin, nedaplatin, nedocromil, nefazodone hydrochloride, neflumozide hydrochloride, nefopam hydrochloride, nelezaprine maleate, nemazoline hydrochloride, nemorubicin, neomycin palmitate, neostigmine bromide, neridronic acid, netilmicin sulfate, neutral endopeptidase, neutramycin, nevirapine, nexeridine hydrochloride, niacin, nibroxane, nicardipine hydrochloride, nicergoline, niclosamide, nicorandil, nicotinyl alcohol, nicotine (commercially available as NICOTROL® NS from Pfizer, Inc.), nifedipine, nifirmerone, nifluridide, nifuradene, nifuraldezone, nifuratel, nifuratrone, nifurdazil, nifurimide, nifurpirinol, nifurquinazol, nifurthiazole, nilutamide, nilvadipine, nimazone, nimodipine, niperotidine, niravoline, niridazole, nisamycin, nisbuterol mesylate, nisin, nisobamate, nisoldipine, nisoxetine, nisterime acetate, nitarsone, nitazoxamide, nitecapone, nitrafudam hydrochloride, nitralamine hydrochloride, nitramisole hydrochloride, nitrazepam, nitrendipine, nitrocycline, nitrodan, nitrofurantoin, nitrofurazone, nitroglycerin, nitromersol, nitromide, nitromifene citrate, nitrous oxide, nitroxide antioxidant, nitrullyn, nivazol, nivimedone sodium, nizatidine, noberastine, nocodazole, nogalamycin, nolinium bromide, nomifensine maleate, noracymethadol hydrochloride, norbolethone, norepinephrine bitartrate, norethindrone, norethynodrel, norfloxacin, norflurane, norgestimate, norgestomet, norgestrel, nortriptyline hydrochloride, noscapine, novobiocin sodium, N-substituted benzaimides, nufenoxole, nylestriol, nystatin, 06-benzylguanine, obidoxime chloride, ocaperidone, ocfentanil hydrochloride, ocinaplon, octanoic acid, octazamide, octenidine hydrochloride, octodrine, octreotide, octriptyline phosphate, ofloxacin, oformine, okicenone, olanzapine (commercially available as ZYPREXA® from Eli Lilly and Company), oligonucleotides, olopatadine, olprinone, olsalazine, olsalazine sodium, olvanil, omeprazole, onapristone, ondansetron, ontazolast, oocyte maturation inhibitor, opipramol hydrochloride, oracin, orconazole nitrate, orgotein, orlislat, ormaplatin, ormetoprim, ornidazole, orpanoxin, orphenadrine citrate, osaterone, otenzepad, oxacillin sodium, oxagrelate, oxaliplatin, oxamarin hydrochloride, oxamisole, oxamniquine, oxandrolone, oxantel pamoate, oxaprotiline hydrochloride, oxaprozin, oxarbazole, oxatomide, oxaunomycin, oxazepam, oxcarbazepine, oxendolone, oxethazaine, oxetorone fumarate, oxfendazole, oxfenicine, oxibendazole, oxiconazole, oxidopamine, oxidronic acid, oxifungin hydrochloride, oxilorphan, oximonam, oximonam sodium, oxiperomide, oxiracetam, oxiramide, oxisuran, oxmetidine hydrochloride, oxodipine, oxogestone phenpropionate, oxolinic acid, oxprenolol hydrochloride, oxtriphylline, oxybutynin chloride, oxychlorosene, oxycodone, oxymetazoline hydrochloride, oxymetholone, oxymorphone hydrochloride, oxypertine, oxyphenbutazone, oxypurinol, oxytetracycline, oxytocin, ozagrel, ozolinone, paclitaxel, palauamine, paldimycin, palinavir, paliperidone (commercially available as INVEGA® from Janssen Pharmaceuticals, Inc.), paliperidone palmitate (commercially available as INVEGA®SUSTENNA® from Janssen Pharmaceuticals, Inc.), palmitoylrhizoxin, palmoxirate sodium, pamaqueside, pamatolol sulfate, pamicogrel, pamidronate disodium, pamidronic acid, panadiplon, panamesine, panaxytriol, pancopride, pancuronium bromide, panipenem, pannorin, panomifene, pantethine, pantoprazole, papaverine hydrochloride, parabactin, parachlorophenol, paraldehyde, paramethasone acetate, paranyline hydrochloride, parapenzolate bromide, pararosaniline pamoate, parbendazole, parconazole hydrochloride, paregoric, pareptide sulfate, pargyline hydrochloride, parnaparin sodium, paromomycin sulfate, paroxetine (commercially available as PAXIL® from GlaxoSmithKlein), parthenolide, partricin, paulomycin, pazelliptine, pazinaclone, pazoxide, pazufloxacin, pefloxacin, pegaspargase, pegorgotein, pelanserin hydrochloride, peldesine, peliomycin, pelretin, pelrinone hydrochloride, pemedolac, pemerid nitrate, pemetrexed, pemirolast, pemoline, penamecillin, penbutolol sulfate, penciclovir, penfluridol, penicillin G benzathine, penicillin G potassium, penicillin G procaine, penicillin G Sodium, penicillin V, penicillin V benzathine, penicillin V hydrabamine, penicillin V potassium, pentabamate, pentaerythritol tetranitrate, pentafuside, pentamidine, pentamorphone, bentamustine, pentapiperium methylsulfate, pentazocine, pentetic acid, pentiapine maleate, pentigetide, pentisomicin, pentizidone sodium, pentobarbital, pentomone, pentopril, pentosan, pentostatin, pentoxifylline, pentrinitrol, pentrozole, peplomycin sulfate, pepstatin, perflubron, perfof amide, perfosfamide, pergolide, perhexyline maleate, perillyl alcohol, perindopril, perindoprilat, perlapine, permethrin, perospirone, perphenazine, phenacemide, phenaridine, phenazinomycin, phenazopyridine hydrochloride, phenbutazone sodium glycerate, phencarbamide, phencyclidine hydrochloride, phendimetrazine tartrate, phenelzine sulfate, phenmetrazine hydrochloride, phenobarbital, phenoxybenzamine hydrochloride, phenprocoumon, phenserine, phensuccinal, phensuximide, phentermine, phentermine hydrochloride, phentolamine mesilate, phentoxifylline, phenyl aminosalicylate, phenylacetate, phenylalanine, phenylalanyl ketoconazole, phenylbutazone, phenylephrine hydrochloride, phenylpropanolamine hydrochloride, phenylpropanolamine polistirex, phenyramidol hydrochloride, phenyloin, phosphatase inhibitors, physostigmine, picenadol, picibanil, picotrin diolamine, picroliv, picumeterol, pidotimod, pifamine, pilocarpine, pilsicamide, pimagedine, pimetine hydrochloride, pimilprost, pimobendan, pimozide, pinacidil, pinadoline, pindolol, pinnenol, pinocebrin, pinoxepin hydrochloride, pioglitazone (commercially available as ACTOS® from Takeda Pharmaceuticals), pipamperone, pipazethate, pipecuronium bromide, piperacetazine, piperacillin sodium, piperamide maleate, piperazine, pipobroman, piposulfan, pipotiazine palmitate, pipoxolan hydrochloride, piprozolin, piquindone hydrochloride, piquizil hydrochloride, piracetam, pirandamine hydrochloride, pirarubicin, pirazmonam sodium, pirazolac, pirbenicillin sodium, pirbuterol acetate, pirenperone, pirenzepine hydrochloride, piretanide, pirfenidone, piridicillin sodium, piridronate sodium, piriprost, piritrexim, pirlimycin hydrochloride, pirlindole, pirmagrel, pirmenol hydrochloride, pirnabine, piroctone, pirodavir, pirodomast, pirogliride tartrate, pirolate, pirolazamide, piroxantrone hydrochloride, piroxicam, piroximone, pirprofen, pirquinozol, pirsidomine, prenylamine, pitavastatin (commercially available as LIVALOA® from Eli Lilly and Company), pituitary, posterior, pivampicillin hydrochloride, pivopril, pizotyline, placetin A, platinum compounds, platinum-triamine complex, plicamycin, plomestane, pobilukast edamine, podofilox, poisonoak extract, poldine methylsulfate, poliglusam, polignate sodium, polymyxin B sulfate, polythiazide, ponalrestat, porfimer sodium, porfiromycin, potassium chloride, potassium iodide, potassium permanganate, povidone-iodine, practolol, pralidoxime chloride, pramiracetam hydrochloride, pramoxine hydrochloride, pranolium chloride, prasugrel (commercially available as EFFIENT® from Eli Lilly and Company), pravadoline maleate, pravastatin, prazepam, prazosin, prazosin hydrochloride, prednazate, prednicarbate, prednimustine, prednisolone, prednisone, prednival, pregabalin (commercially available as LYRICA® from Pfizer, Inc.), pregnenolone succiniate, prenalterol hydrochloride, pridefine hydrochloride, prifelone, prilocalne hydrochloride, prilosec, primaquine phosphate, primidolol, primidone, prinivil, prinomide tromethamine, prinoxodan, prizidilol hydrochloride, proadifen hydrochloride, probenecid, probicromil calcium, probucol, procainamide hydrochloride, procaine hydrochloride, procarbazine hydrochloride, procaterol hydrochloride, prochlorperazine, procinonide, proclonol, procyclidine hydrochloride, prodilidine hydrochloride, prodolic acid, prof adol hydrochloride, progabide, progesterone, proglumide, proinsulin human, proline, prolintane hydrochloride, promazine hydrochloride, promethazine hydrochloride, propafenone hydrochloride, propagermanium, propanidid, propantheline bromide, proparacaine hydrochloride, propatyl nitrate, propentofylline, propenzolate hydrochloride, propikacin, propiomazine, propionic acid, propionylcarnitine, propiram, propiram+paracetamol, propiverine, propofol, propoxycaine hydrochloride, propoxyphene hydrochloride, propranolol hydrochloride, propulsid, propyl bis-acridone, propylhexedrine, propyliodone, propylthiouracil, proquazone, prorenoate potassium, proroxan hydrochloride, proscillaridin, prostalene, prostratin, protamine sulfate, protegrin, protirelin, protosufloxacin, protriptyline hydrochloride, proxazole, proxazole citrate, proxicromil, proxorphan tartrate, prulifloxacin, pseudoephedrine hydrochloride, desloratadine/pseudoephedrine sulfate (commercially available as CLARINEX-D® from Merck), puromycin, purpurins, pyrabrom, pyrantel, pamoate, pyrazinamide, pyrazofurin, pyrazoloacridine, pyridostigmine bromide, pyrilamine maleate, pyrimethamine, pyrinoline, pyrithione sodium, pyrithione zinc, pyrovalerone hydrochloride, pyroxamine maleate, pyrrocaine, pyrroliphene hydrochloride, pyrroinitrin, pyrvinium pamoate, quadazocine mesylate, quazepam, quazinone, quazodine, quazolast, quetiapine (commercially available as SEROQUEL® available from AstraZenica), quiflapon, quinagolide, quinaldine blue, quinapril, quinaprilat, quinazosin hydrochloride, quinbolone, quinctolate, quindecamine acetate, quindonium bromide, quinelorane hydrochloride, quinestrol, quinfamide, quingestanol acetate, quingestrone, quinidine gluconate, quinielorane hydrochloride, quinine sulfate, quinpirole hydrochloride, quinterenol sulfate, quinuclium bromide, quinupristin, quipazine maleate, rabeprazole sodium, racephenicol, racepinephrine, raf antagonists, rafoxamide, ralitoline, raloxifene, raltitrexed, ramatroban, ramipril, ramoplanin, ramosetron, ranelic acid, ranimycin, ranitidine, ranolazine, rauwolfia serpentina, recainam, recainam hydrochloride, reclazepam, regavirumab, regramostim, relaxin, relomycin, remacemide hydrochloride, remifentanil hydrochloride, remiprostol, remoxipride, repirinast, repromicin, reproterol hydrochloride, reserpine, resinferatoxin, resorcinol, retelliptine demethylated, reticulon, reviparin sodium, revizinone, rhenium re 186 etidronate, rhizoxin, ribaminol, ribavirin, riboprine, ribozymes, ricasetron, ridogrel, rifabutin, rifametane, rifamexil, rifamide, rifampin, rifapentine, rifaximin, retinamide, rilopirox, riluzole, rimantadine, rimcazole hydrochloride, rimexolone, rimiterol hydrobromide, rimoprogin, riodipine, rioprostil, ripazepam, ripisartan, risedronate sodium, risedronic acid, risocaine, risotilide hydrochloride, rispenzepine, risperdal, risperidone, ritanserin, ritipenem, ritodrine, ritolukast, ritonavir, rizatriptan benzoate, rocastine hydrochloride, rocuronium bromide, rodocaine, roflurane, rogletimide, rohitukine, rokitamycin, roletamicide, rolgamidine, rolicyprine, rolipram, rolitetracycline, rolodine, romazarit, romurtide, ronidazole, ropinirole (commercially available as REQUIP® from GlaxoSmithKline), ropitoin hydrochloride, ropivacaine, ropizine, roquinimex, rosaramicin, rosoxacin, rotoxamine, rosuvastatin (commercially available as CRESTOR® available from AstraZeneca), roxaitidine, roxarsone, roxindole, roxithromycin, rubiginone B1, ruboxyl, rufloxacin, rupatidine, rutamycin, ruzadolane, sabeluzole, safingol, safironil, saintopin, salbutamol, salcolex, salethamide maleate, salicyl alcohol, salicylamide, salicylate meglumine, salicylic acid, salmeterol, salnacediin, salsalate, sameridine, sampatrilat, sancycline, sanfetrinem, sanguinarium chloride, saperconazole, saprisartan, sapropterin, saquinavir, sarafloxacin hydrochloride, saralasin acetate, SarCNU, sarcophytol A, sargramostim, sarmoxicillin, sarpicillin, sarpogrelate, saruplase, saterinone, satigrel, satumomab pendetide, schick test control, scopafungin, scopolamine hydrobromide, scrazaipine hydrochloride, sdi 1 mimetics, secalciferol, secobarbital, seelzone, seglitide acetate, selegiline, selegiline hydrochloride, selenium sulfide, selenomethionine se 75, selfotel, sematilide, semduramicin, semotiadil, semustine, sense oligonucleotides, sepazonium chloride, seperidol hydrochloride, seprilose, seproxetine hydrochloride, seractide acetate, sergolexole maleate, serine, sermetacin, sermorelin acetate, sertaconazole, sertindole, sertraline, setiptiline, setoperone, sevirumab, sevoflurane, sezolamide, sibopirdine, sibutramine hydrochloride, signal transduction inhibitors, silandrone, sildenafil (commercially available as VIAGRA® from Pfizer Inc.), silipide, silteplase, silver nitrate, simendan, simtrazene, simvastatin (commercially available as ZOCOR® from Merck), sincalide, sinefungin, sinitrodil, sinnabidol, sipatrigine, sirolimus, sisomicin, sitogluside, sizofuran, sobuzoxane, sodium amylosulfate, sodium iodide 1123, sodium nitroprusside, sodium oxybate, sodium phenylacetate, sodium salicylate, solverol, solypertine tartrate, somalapor, somantadine hydrochloride, somatomedin B, somatomedin C, somatrem, somatropin, somenopor, somidobove, sonermin, sorbinil, sorivudine, sotalol, soterenol hydrochloride, sparfloxacin, sparfosate sodium, sparfosic acid, sparsomycin, sparteine sulfate, spectinomycin hydrochloride, spicamycin D, spiperone, spiradoline mesylate, spiramycin, spirapril hydrochloride, spiraprilat, spirogermanium hydrochloride, spiromustine, spironolactone, spiroplatin, spiroxasone, splenopentin, spongistatin 1, sprodiamide, squalamine, stallimycin hydrochloride, stannous pyrophosphate, stannous sulfur colloid, stanozolol, statolon, staurosporine, stavudine, steffimycin, stenbolone acetate, stepronin, stilbazium iodide, stilonium iodide, stipiamide, stiripentol, stobadine, streptomycin sulfate, streptonicozid, streptonigrin, streptozocin, stromelysin inhibitors, strontium chloride Sr 89, succibun, succimer, succinylcholine chloride, sucralfate, sucrosof ate potassium, sudoxicam, sufentanil, sufotidine, sulazepam, sulbactam pivoxil, sulconazole nitrate, sulfabenz, sulfabenzamide, sulfacetamide, sulfacytine, sulfadiazine, sulfadoxine, sulfalene, sulfamerazine, sulfameter, sulfamethazine, sulfamethizole, sulfamethoxazole, sulfamonomethoxine, sulfamoxole, sulfanilate zinc, sulfanitran, sulfasalazine, sulfasomizole, sulfazamet, sulfinalol hydrochloride, sulfinosine, sulfinpyrazone, sulfisoxazole, sulfomyxin, sulfonterol hydrochloride, sulfoxamine, sulinldac, sulmarin, sulnidazole, suloctidil, sulofenur, sulopenem, suloxifen oxalate, sulpiride, sulprostone, sultamicillin, sulthiame, sultopride, sulukast, sumarotene, sumatriptan, suncillin sodium, suproclone, suprofen, suradista, suramin, surfomer, suricamide maleate, suritozole, suronacrine maleate, suxemerid sulfate, swainsonine, symakalim, symclosene, symetine hydrochloride, synthetic glycosaminoglycans, tadalafil (commercially available as CIALIS® and ACIRCA® from Eli Lilly and Company), taciamine hydrochloride, tacrine hydrochloride, tacrolimus, talampicillin hydrochloride, taleranol, talisomycin, tallimustine, talmetacin, talniflumate, talopram hydrochloride, talosalate, tametraline hydrochloride, tamoxifen (commercially available as NOLVADEX® from AstraZeneca), tampramine fumarate, tamsulosin hydrochloride, tandamine hydrochloride, tandospirone, tapgen, taprostene, tasosartan, tauromustine, taxane, taxoid, tazadolene succinate, tazanolast, tazarotene, tazifylline hydrochloride, tazobactam, tazofelone, tazolol hydrochloride, tebufelone, tebuquine, technetium Tc 99 m bicisate, teclozan, tecogalan sodium, teecleukin, teflurane, tegafur, tegretol, teicoplanin, telenzepine, tellurapyrylium, telmesteine, telmisartan, telomerase inhibitors, teloxantrone hydrochloride, teludipine hydrochloride, temafloxacin hydrochloride, tematropium methyl sulfate, temazepam, temelastine, temocapril, temocillin, temoporfin, temozolomide, tenofovir, tenidap, teniposide, tenosal, tenoxicam, tepirindole, tepoxalin, teprotide, terazosin, terbinafine, terbutaline sulfate (commercially available as BRICANYL® from AstraZeneca), terconazole, terfenadine, terflavoxate, terguride, teriparatide acetate, terlakiren, terlipressin, terodiline, teroxalene hydrochloride, teroxirone, tertatolol, tesicam, tesimide, testolactone, testosterone, tetracaine, tetrachlorodecaoxide, tetracycline, tetrahydrozoline hydrochloride, tetramisole hydrochloride, tetrazolast meglumine, tetrazomine, tetrofosmin, tetroquinone, tetroxoprim, tetrydamine, thaliblastine, thalidomide, theofibrate, theophylline, thiabendazole, thiamiprine, thiamphenicol, thiamylal, thiazesim hydrochloride, thiazinamium chloride, thiazolidinedione, thiethylperazine, thimerfonate sodium, thimerosal, thiocoraline, thiofedrine, thioguanine, thiomarinol, thiopental sodium, thioperamide, thioridazine, thiotepa, thiothixene, thiphenamil hydrochloride, thiphencillin potassium, thiram, thozalinone, threonine, thrombin, thrombopoietin, thrombopoietin mimetic, thymalfasin, thymopoietin receptor agonist, thymotrinan, thyromedan hydrochloride, thyroxine 1 125, thyroxine 1 131, tiacrilast, tiacrilast sodium, tiagabine, tiamenidine, tianeptine, tiapafant, tiapamil hydrochloride, tiaramide hydrochloride, tiazofurin, tibenelast sodium, tibolone, tibric acid, ticabesone propionate, ticarbodine, ticarcillin cresyl sodium, ticlatone, ticlopidine, ticrynafen, tienoxolol, tifurac sodium, tigemonam dicholine, tigestol, tiletamine hydrochloride, tilidine hydrochloride, tilisolol, tilnoprofen arbamel, tilorone hydrochloride, tiludronate disodium, tiludronic acid, timefurone, timobesone acetate, timolol, tin ethyl etiopurpurin, tinabinol, timidazole, tinzaparin sodium, tioconazole, tiodazosin, tiodonium chloride, tioperidone hydrochloride, tiopinac, tiospirone hydrochloride, tiotidine, tiotropium bromide, tioxidazole, tipentosin hydrochloride, tipredane, tiprenolol hydrochloride, tiprinast meglumine, tipropidil hydrochloride, tiqueside, tiquinamide hydrochloride, tirandalydigin, tirapazamine, tirilazad, tirofiban, tiropramide, titanocene dichloride, tixanox, tixocortol pivalate, tizanidine hydrochloride, tobramycin, tocamide, tocamphyl, tofenacin hydrochloride, tolamolol, tolazamide, tolazoline hydrochloride, tolbutamide, tolcapone, tolciclate, tolfamide, tolgabide, lamotrigine, tolimidone, tolindate, tolmetin, tolnaftate, tolpovidone 1 131, tolpyrramide, tolrestat, tomelukast, tomoxetine hydrochloride, tonazocine mesylate, topiramate, topotecan, topotecan hydrochloride, topsentin, topterone, toquizine, torasemide, toremifene, torsemide, tosifen, tosufloxacin, totipotent stem cell factor, tracazolate, trafermin, tralonide, tramadol hydrochloride, tramazoline hydrochloride, trandolapril, tranexamic acid, tranilast, transcamide, translation inhibitors, trastuzumab (commercially available as HERCEPTIN® from Genentech), traxanox, trazodone hydrochloride, trazodone-hcl, trebenzomine hydrochloride, trefentanil hydrochloride, treloxinate, trepipam maleate, trestolone acetate, tretinoin, triacetin, triacetyluridine, triafungin, triamcinolone, triampyzine sulfate, triamterene, triazolam, tribenoside, tricaprilin, tricetamide, trichlormethiazide, trichohyalin, triciribine, tricitrates, triclofenol piperazine, triclofos sodium, triclonide, trientine, trifenagrel, triflavin, triflocin, triflubazam, triflumidate, trifluoperazine hydrochloride, trifluperidol, triflupromazine, triflupromazine hydrochloride, trifluridine, trihexyphenidyl hydrochloride, trilostane, trimazosin hydrochloride, trimegestone, trimeprazine tartrate, trimethadione, trimethaphan camsylate, trimethobenzamide hydrochloride, trimethoprim, trimetozine, trimetrexate, trimipramine, trimoprostil, trimoxamine hydrochloride, triolein 1 125, triolein 1 131, trioxifene mesylate, tripamide, tripelennamine hydrochloride, triprolidine hydrochloride, triptorelin, trisulfapyrimidines, troclosene potassium, troglitazone, trolamine, troleandomycin, trombodipine, trometamol, tropanserin hydrochloride, tropicamide, tropine ester, tropisetron, trospectomycin, trovafloxacin, trovirdine, tryptophan, tuberculin, tubocurarine chloride, tubulozole hydrochloride, tucarcsol, tulobuterol, turosteride, tybamate, tylogenin, tyropanoate sodium, tyrosine, tyrothricin, tyrphostins, ubenimex, uldazepam, undecylenic acid, uracil mustard, urapidil, urea, uredepa, uridine triphosphate, urofollitropin, urokinase, ursodiol, valaciclovir, valine, valnoctamide, valproate sodium, valproic acid, valsartan (commercially available as DIOVAN® from Novartis Pharmaceuticals), vamicamide, vanadeine, vancomycin, vaminolol, vapiprost hydrochloride, vapreotide, vardenafil (commercially available as LEVITRA® from GlaxoSmithKline), variolin B, vasopressin, vecuronium bromide, velaresol, velnacrine maleate, venlafaxine, veradoline hydrochloride, veramine, verapamil hydrochloride, verdins, verilopam hydrochloride, verlukast, verofylline, veroxan, verteporfin, vesnarinone, vexibinol, vidarabine, vigabatrin, viloxazine hydrochloride, vinblastine sulfate, vinburnine citrate, vincofos, vinconate, vincristine sulfate, vindesine, vindesine sulfate, vinepidine sulfate, vinglycinate sulfate, vinleurosine sulfate, vinorelbine, vinpocetine, vintoperol, vinxaltine, vinzolidine sulfate, viprostol, virginiamycin, viridofulvin, viroxime, vitaxin, volazocine, voriconazole, vorozole, voxergolide, warfarin sodium, xamoterol, xanomeline, xanoxate sodium, xanthinol niacinate, xemilofiban, xenalipin, xenbucin, xilobam, ximoprofen, xipamide, xorphanol mesylate, xylamidine tosylate, xylazine hydrochloride, xylometazoline hydrochloride, xylose, yangambin, zabicipril, zacopride, zafirlukast, zalcitabine, zaleplon, zalospirone, zaltidine hydrochloride, zaltoprofen, zanamivir, zankiren, zanoterone, zantac, zarirlukast, zatebradine, zatosetron, zatosetron maleate, zenarestat, zenazocine mesylate, zeniplatin, zeranol, zidometacin, zidovudine, zifrosilone, zilantel, zilascorb, zileuton, zimeldine hydrochloride, zinc undecylenate, zindotrine, zinoconazole hydrochloride, zinostatin, zinterol hydrochloride, zinviroxime, ziprasidone, zobolt, zofenopril calcium, zofenoprilat, zolamine hydrochloride, zolazepam hydrochloride, zoledronie acid, zolertine hydrochloride, zolmitriptan, zolpidem, zomepirac sodium, zometapine, zoniclezole hydrochloride, zonisamide, zopiclone, zopolrestat, zorbamyciin, zorubicin hydrochloride, zotepine, zucapsaicin, JTT-501 (PNU-182716) (reglitazar), AR-H039122, MCC-555 (netoglitazone), AR-H049020 (tesaglitazar), CS-011 (CI-1037), GW-409544x, KRP-297, RG-12525, BM-15.2054, CLX-0940, CLX-0921, DRF-2189, GW-1929, GW-9820, LR-90, LY-510929, NIP-221, NIP-223, JTP-20993, LY 29311 Na, FK 614, BMS 298585, R 483, TAK 559, DRF 2725 (ragaglitazar), L-686398, L-168049, L-805645, L-054852, demethyl asteriquinone B1 (L-783281), L-363586, KRP-297, P32/98, CRE-16336, EML-1625, pharmaceutically acceptable salts thereof (e.g., Zn, Fe, Mg, K, Na, F, Cl, Br, I, acetate, diacetate, nitrate, nitrite, sulfate, sulfite, phosphate, and phosphite salts), pharmaceutically acceptable forms thereof with acid associates (e.g. HCl), and any combination thereof.

A typical dosage of agents (active agents (e.g., active pharmaceuticals and prodrugs of active pharmaceuticals), removal agents, and tracking agents) might range from about 0.001 mg/kg to about 1000 mg/kg, preferably from about 0.01 mg/kg to about 100 mg/kg, and more preferably from about 0.10 mg/kg to about 20 mg/kg, relative to weight of the patient. In some embodiments, active pharmaceuticals and prodrugs of active pharmaceuticals may be used alone or in combination with other agents. One skilled in the art should understand the dose and/or combination of agents should be chosen so as to minimize adverse interactions. Further, one skilled in the art should recognize that controlled release vehicles of the present invention may allow for combinations of agents not previously realized by exploiting the potential for complex macrostructures and the plurality of possible release rates.

Suitable antibiotics for use in conjunction with the present invention may include, but are not limited to, to β-lactam antibiotics (e.g., benzathine penicillin, benzylpenicillin (penicillin G), phenoxymethylpenicillin (penicillin V), procaine penicillin, methicillin, oxacillin, nafcillin, cloxacillin, dicloxacillin, flucloxacillin, temocillin, amoxicillin, ampicillin, co-amoxiclav (amoxicillin+clavulanic acid), azlocillin, carbenicillin, ticarcillin, mezlocillin, piperacillin, cephalosporin, cephalexin, cephalothin, cefazolin, cefaclor, cefuroxime, cefamandole, cefotetan, cefoxitin, ceftriaxone, cefotaxime, cefpodoxime, cefixime, ceftazidime, cefepime, cefpirome, carbapenem, imipenem (with cilastatin), meropenem, ertapenem, faropenem, doripenem, aztreonam (commercially available as AZACTAM® from Bristol-Myers Squibb), tigemonam, nocardicin A, tabtoxinine-β-lactam, clavulanic acid, tazobactam, and sulbactam); aminoglycoside antibiotics (e.g., aminoglycoside, amikacin, apramycin, arbekacin, astromicin, bekanamycin, capreomycin, dibekacin, dihydrostreptomycin, elsamitrucin, G418, gentamicin, hygromycin B, isepamicin, kanamycin, kasugamycin, micronomicin, neomycin, netilmicin, paromomycin sulfate, ribostamycin, sisomicin, streptoduocin, streptomycin, tobramycin, verdamicin; sulfonamides such as sulfamethoxazole, sulfisomidine (also known as sulfaisodimidine), sulfacetamide, sulfadoxine, dichlorphenamide (DCP), and dorzolamide); quinolone antibiotics (e.g., cinobac, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, sparfloxacin, temafloxacin, tosufloxacin, clinafloxacin, gatifloxacin, gemifloxacin, moxifloxacin, sitafloxacin, trovafloxacin, prulifloxacin, garenoxacin, and delafloxacin); oxazolidone antibiotics (e.g., linezolid, torezolid, eperezolid, posizolid, and radezolid), and any combination thereof.

Suitable antifungals for use in conjunction with the present invention may include, but are not limited to, polyene antifungals (e.g., natamycin, rimocidin, filipin, nystatin, amphotericin B, candicin, and hamycin; imidazole antifungals such as miconazole (commercially available as MICATIN® from WellSpring Pharmaceutical Corporation), ketoconazole (commercially available as NIZORAL® from McNeil consumer Healthcare), clotrimazole (commercially available as LOTRAMIN® and LOTRAMIN AF® available from Merck and CANESTEN® available from Bayer), econazole, omoconazole, bifonazole, butoconazole, fenticonazole, isoconazole, oxiconazole, sertaconazole (commercially available as ERTACZO® from OrthoDematologics), sulconazole, and tioconazole; triazole antifungals such as fluconazole, itraconazole, isavuconazole, ravuconazole, posaconazole, voriconazole, terconazole, and albaconazole), thiazole antifungals (e.g., abafungin), allylamine antifungals (e.g., terbinafine (commercially available as LAMISIL® from Novartis Consumer Health, Inc.), naftifine (commercially available as NAFTIN® available from Merz Pharmaceuticals), and butenafine (commercially available as LOTRAMIN ULTRA® from Merck), echinocandin antifungals (e.g., anidulafungin, caspofungin, and micafungin), polygodial, benzoic acid, ciclopirox, tolnaftate (e.g., commercially available as TINACTIN® from MDS Consumer Care, Inc.), undecylenic acid, flucytosine, 5-fluorocytosine, griseofulvin, haloprogin, and any combination thereof.

Suitable active biologicals for use in conjunction with the present invention may include, but are not limited to, hormones (synthetic or natural and patient derived or otherwise), DNAs (synthetic or natural and patient derived or otherwise), RNAs (synthetic or natural and patient derived or otherwise), siRNAs (synthetic or natural and patient derived or otherwise), proteins and peptides (e.g., albumin, atrial natriuretic factor, renin, superoxide dismutase, a 1-antitrypsin, lung surfactant proteins, bacitracin, bestatin, cydosporine, delta sleep-inducing peptide (DSIP), endorphins, glucagon, gramicidin, melanocyte inhibiting factors, neurotensin, oxytocin, somostatin, terprotide, serum thymide factor, thymosin, DDAVP, dermorphin, Met-enkephalin, peptidoglycan, satietin, thymopentin, fibrin degradation product, des-enkephalin-α-endorphin, gonadotropin releasing hormone, leuprolide, α-MSH, and metkephamid), enzymes, nucleotides, oligionucleotides, antibodies, monoclonal antibodies, growth factors (e.g., epidermal growth factor (EGF), fibroblast growth factors, basic fibroblast growth factor (bFGF), nerve growth factor (NGF), bone derived growth factor (BDGF), transforming growth factors, transforming growth factor-131 (TGF-131), and human growth gormone (hGH)), viral surface antigens (e.g., adenoviruses, epstein-barr virus, hepatitis A virus, hepatitis B virus, herpes viruses, HIV-1, HIV-2, HTLV-III, influenza viruses, Japanese encephalitis virus, measles virus, papilloma viruses, paramyxoviruses, polio virus, rabies virus, rubella virus, vaccinia (smallpox) viruses, and yellow fever virus), bacterial surface antigens (e.g., bordetella pertussis, helicobacter pylorn, clostridium tetani, corynebacterium diphtheria, escherichia coli, haemophilus influenza, klebsiella species, legionella pneumophila, mycobacterium bovis, mycobacterium leprae, mycrobacterium tuberculosis, neisseria gonorrhoeae, neisseria meningitidis, proteus species, pseudomonas aeruginosa, salmonella species, shigella species, staphylococcus aureus, streptococcus pyogenes, vibrio cholera, and yersinia pestis), parasite surface antigens (e.g., plasmodium vivax-malaria, plasmodium falciparum-malaria, plasmodium ovale-malaria, plasmodium malariae-malaria, leishmania tropica-leishmaniasis, leishmania donovani, leishmaniasis, leishmania branziliensis-leishmaniasis, trypanosome rhodescense-sleeping sickness, trypanosoma gambiense-sleeping sickness, trypanosome cruzi-Chagas' disease, schistosoma mansoni-schistosomiasis, schistosomoma haematobium-schistomiasis, schistosoma japonicum-shichtomiasis, trichinella spiralis-trichinosis, stronglyloides duodenale-hookworm, ancyclostoma duodenale-hookworm, necator americanus-hookworm, wucheria bancrofti-filariasis, brugia malaya-filariasis, loa loa-filariasis, dipetalonema perstaris-filariasis, dracuncula medinensis-filariasis, and onchocerca volvulus-filariasis), immunogobulins (e.g., IgG, IgA, IgM, antirabies immunoglobulin, and antivaccinia immunoglobulin), and any combination thereof.

Suitable antitoxins for use in conjunction with the present invention may include, but are not limited to, botulinum antitoxin, diphtheria antitoxin, gas gangrene antitoxin, tetanus antitoxin, and any combination thereof.

Suitable antigents for use in conjunction with the present invention may include, but are not limited to, foot and mouth disease, hormones and growth factors (e.g., follicle stimulating hormone, prolactin, angiogenin, epidermal growth factor, calcitonin, erythropoietin, thyrotropic releasing hormone, insulin, growth hormones, insulin-like growth factors 1 and 2, skeletal growth factor, human chorionic gonadotropin, luteinizing hormone, nerve growth factor, adrenocorticotropic hormone (ACTH), luteinizing hormone releasing hormone (LHRH), parathyroid hormone (PTH), thyrotropin releasing hormone (TRH), vasopressin, cholecystokinin, and corticotropin releasing hormone), cytokines (e.g., interferons, interleukins, colony stimulating factors, and tumor necrosis factors: fibrinolytic enzymes, such as urokinase, kidney plasminogen activator), clotting factors (e.g., Protein C, Factor VIII, Factor IX, Factor VII and Antithrombin III), and any combination thereof.

Suitable cells and cell-like structures for use in conjunction with the present invention may include, but are not limited to, endothelial cells, hepatic cells, myocytes, smooth muscle cells, nerve cells, progenitor cells, stem cells, parthenogenetic stem cell, activated version thereof (e.g., those overexpressing a marker), deactivated version thereof (e.g., those underexpressing a marker), synthetic cells, and the like.

Suitable nutritional supplements for use in conjunction with the present invention may include, but are not limited to, vitamins, minerals, herbs, botanicals, amino acids, steroids, and the like.

Suitable imaging agents for use in conjunction with the present invention may include, but are not limited to, iron oxide, gadolinium ions, iodine, perfluorocarbons, radioisotopes, and the like.

Suitable fluid stabilizers for use in conjunction with the present invention may include, but are not limited to, at least one component of citrate phosphate with dextrose buffer (e.g., stabilizing blood), blood clotting factors, emulsion stabilizers, antifoamers, agar, pectin, and the like, and any combination thereof.

Suitable food agents for use in conjunction with the present invention may include, but are not limited to, caffeine, flavors, aromas, vitamins, minerals, herbs, minerals, antioxidants, calcium propionate, sodium nitrate, sodium nitrite, sulfites, sulfur dioxide, sodium bisulfite, potassium hydrogen sulfite, disodium EDTA, salt, rosemary extract, sugar, vinegar, alcohol, hops, diatomaceous earth, and the like, and any combination thereof.

Suitable nutraceuticals for use in conjunction with the present invention may include, but are not limited to, dietary supplements, botanicals, functional foods and extracts thereof, medicinal foods and extracts thereof, vitamins, minerals, co-enzyme Q, carnitine, multi-mineral formulas, gingseng, gingko biloba, saw palmetto, other plant-based supplements, probiotics, omega-3, canola and other oils, plant stanols, natural sweeteners, mushroom extracts, chocolate, chocolate extracts, grape extracts, berry extracts, super food extracts, quillaja molina extracts, plant extracts, yucca schidigera extract, bran, alanine, beta-carotene, carotenoids, arginin, vitamin A, asparagine, vitamin B-complex, aspartate, vitamin C, leucine, isoleucine, valine, vitamin D, citrulline, vitamin E, cysteine, vitamin K, glutamine, minerals, micro-nutrients, glutamic acid, calcium, glycine, chromium, histidine, copper, lysine, iodine, methionine, iron, ornithine, magnesium, phenylalanine, potassium, proline, selenium, serine, zinc, taurine, threonine, alpha lipoic acid, tryptophan, green tea extracts, tyrosine, essential fatty acids (EFA), whey protein, flax seed oil, and any combination thereof.

Suitable olfactory agents for use in conjunction with the present invention may include, but are not limited to, spices, spice extracts, herb extracts, essential oils, smelling salts, volatile organic compounds, volatile small molecules, methyl formate, methyl acetate, methyl butyrate, ethyl acetate, ethyl butyrate, isoamyl acetate, pentyl butyrate, pentyl pentanoate, octyl acetate, myrcene, geraniol, nerol, citral, citronellal, citronellol, linalool, nerolidol, limonene, camphor, terpineol, alpha-ionone, thujone, benzaldehyde, eugenol, cinnamaldehyde, ethyl maltol, vanilla, anisole, anethole, estragole, thymol, furaneol, methanol, rosemary, lavender, citrus, freesia, apricot blossoms, greens, peach, jasmine, rosewood, pine, thyme, oakmoss, musk, vetiver, myrrh, blackcurrant, bergamot, grapefruit, acacia, passiflora, sandalwood, tonka bean, mandarin, neroli, violet leaves, gardenia, red fruits, ylang-ylang, acacia farnesiana, mimosa, tonka bean, woods, ambergris, daffodil, hyacinth, narcissus, black currant bud, iris, raspberry, lily of the valley, sandalwood, vetiver, cedarwood, neroli, bergamot, strawberry, carnation, oregano, honey, civet, heliotrope, caramel, coumarin, patchouli, dewberry, helonial, bergamot, hyacinth, coriander, pimento berry, labdanum, cassie, bergamot, aldehydes, orchid, amber, benzoin, orris, tuberose, palmarosa, cinnamon, nutmeg, moss, styrax, pineapple, bergamot, foxglove, tulip, wisteria, clematis, ambergris, gums, resins, civet, peach, plum, castoreum, civet, myrrh, geranium, rose violet, jonquil, spicy carnation, galbanum, hyacinth, petitgrain, iris, hyacinth, honeysuckle, pepper, raspberry, benzoin, mango, coconut, hesperides, castoreum, osmanthus, mousse de chene, nectarine, mint, anise, cinnamon, orris, apricot, plumeria, marigold, rose otto, narcissus, tolu balsam, frankincense, amber, orange blossom, bourbon vetiver, opopanax, white musk, papaya, sugar candy, jackfruit, honeydew, lotus blossom, muguet, mulberry, absinthe, ginger, juniper berries, spicebush, peony, violet, lemon, lime, hibiscus, white rum, basil, lavender, balsamics, fo-ti-tieng, osmanthus, karo karunde, white orchid, calla lilies, white rose, rhubrum lily, tagetes, ambergris, ivy, grass, sering a, spearmint, clary sage, cottonwood, grapes, brimbelle, lotus, cyclamen, orchid, glycine, tiare flower, ginger lily, green osmanthus, passion flower, blue rose, bay rum, cassie, African tagetes, Anatolian rose, Auvergne narcissus, British broom, British broom chocolate, Bulgarian rose, Chinese patchouli, Chinese gardenia, Calabrian mandarin, Comoros Island tuberose, Ceylonese cardamom, Caribbean passion fruit, Damascena rose, Georgia peach, white Madonna lily, Egyptian jasmine, Egyptian marigold, Ethiopian civet, Farnesian cassie, Florentine iris, French jasmine, French jonquil, French hyacinth, Guinea oranges, Guyana wacapua, Grasse petitgrain, Grasse rose, Grasse tuberose, Haitian vetiver, Hawaiian pineapple, Israeli basil, Indian sandalwood, Indian Ocean vanilla, Italian bergamot, Italian iris, Jamaican pepper, May rose, Madagascar ylang-ylang, Madagascar vanilla, Moroccan jasmine, Moroccan rose, Moroccan oakmoss, Moroccan orange blossom, Mysore sandalwood, Oriental rose, Russian leather, Russian coriander, Sicilian mandarin, South African marigold, South American tonka bean, Singapore patchouli, Spanish orange blossom, Sicilian lime, Reunion Island vetiver, Turkish rose, That benzoin, Tunisian orange blossom, Yugoslavian oakmoss, Virginian cedarwood, Utah yarrow, West Indian rosewood, and the like, and any combination thereof.

Suitable flavorants for use in conjunction with the present invention may include, but are not limited to, tobacco, menthol, cloves, cherry, chocolate, orange, mint, mango, vanilla, cinnamon, and the like. Such flavorants may, in some embodiments, be provided by menthol, anethole (licorice), anisole, limonene (citrus), eugenol (clove), a flavorant associated with an olfactory agent described herein, and the like, and any combination thereof.

Suitable plant agents for use in conjunction with the present invention may include, but are not limited to, herbicides, fungicides, insecticides, bactericides, nitrogen sources, phosphorous sources, potassium sources, calcium sources, magnesium sources, sulfur sources, boron sources, chlorine sources, copper sources, iron sources, manganese sources, molybdenum sources, zinc sources, saltpeter, and the like, and any combination thereof.

Suitable chemical-reaction agents for use in conjunction with the present invention may include, but are not limited to, positive catalysts, inhibitors, and the like, and any combination thereof.

As used herein, the term “insect repellent” refers to both insect repellents and insecticides. One skilled in the art with the benefit of this disclosure should understand that because controlled release vehicles described herein, in some embodiments, are designed to be administered to a patient, insect repellents should be chosen that are compatible with such a desired administration technique. Suitable insect repellents for use in conjunction with the present invention may include, but are not limited to, natural repellents (e.g., essential oils, citronella, sodium laurel sulfate, cedar, neem, clove, thyme, lavender, eucalyptus, peppermint, lemongrass, garlic, capsaicin, sabadillia, rotenone, nicotine, and pyrethrum), synthetic repellents (e.g., N,N-dimethyl-meta-toluamide (DEET), dichlorodiphenyltrichloroethane (DDT), organophosphate-based insecticides, pyrethroids, picaridin, boric acid, cyfluthrin, deltamethrin, fenthion, propoxur, sevin, dinotefuran, acephate, chlorophyrifos, diazinon, horticultural oil, malathion, and methoxyclor), insect controlling pheromones, and the like, and any combination thereof. Suitable insecticides for use in conjunction with the present invention may include, but are not limited to, acid copper chromate (ACC), acetamiprid, bifenazate, chlorantraniliprole, chlorfenapyr, clothianidin, dinotefuran, ethiprole, flubendiamide, flufenoxuron, imiprothrin, indoxacarb, metrafenone, nicarbazin, n-methylneodecanamide, phosphine, pirimicarb, pyridalyl, spinetoram, spinosad, spirodiclofen, spirotetramat, tebufenpyrad, thiacloprid, pyrethrin, allethrin, prallethrin, furamethrin, phenothrin, permethrin, imidacloprid, pyriproxyfen silafluofen, hinokitiol, isopropylmethyl phenol, 5-chloro-2-trifluoromethanesulfonamide methyl benzoate, taufluvalinate, flumethrin, trans-cyfluthrin, kadethrin, bioresmethrin, tetramethrin, empenthrin, cyphenothrin, bioallethrin, an oxadiazine derivative, a chloronicotinyl, a nitroguanidine, a pyrrol, a pyrazone, a diacylhydrazine, a triazole, a biological/fermentation product, a phenyl pyrazole, an organophosphate, a carbamate, a pyrethrin, d-trans allethrin, esbiol, esbiothrin, pynamin forte, n-octyl bicycloheptene dicarboximide, and the like, and any combination thereof. Further, an insect repellent may be utilized, in some embodiments, in conjunction with an insect repellent synergist, a chemical or biological compound that interferes with an insect's ability to mitigate the effects of an insect repellent. Suitable insect repellent synergists may include, but are not limited to, piperonyl butoxide, dietholate, sesamex, sulfoxide, butcarpolate, sesamolin, jiajizengxiaolin, octachlorodipropylether, piperonyl cyclonene, piprotal, propylisome, and any combination thereof. In some embodiments, an insect repellent, and preferably an insect repellant that comprises an insecticide, may be used in conjunction with compounds that attracts insect to a controlled release vehicle of the present invention, including, but not limited to, any suitable olefactory agent described herein.

The following examples of preferred or representative embodiments are given. In no way should the following examples be read to limit, or to define, the scope of the invention.

### Examples

Five samples of ethylene vinyl acetate (“EVA”) copolymer having 28% vinyl acetate content and a melt flow index of 25 were irradiated in pellet form with varying radiation doses from an electron beam source to achieve partially crosslinked EVA copolymer pellets. After having been irradiated, the melt flow index of the partially crosslinked EVA copolymer was measured by ASTM D1238 at 190° C. using a load of 2160 g (2.16 kg), the results of which are shown in Table 1.

TABLE 1

| Radiation Dose | Melt Flow Index |
|---|---|
| 15 kGy | 0.359 |
| 20 kGy | 0.04 |
| 25 kGy | 0.03 |
| 30 kGy | 0.016 |
| 35 kGy | 0 |

This example demonstrates that EVA copolymer can be irradiated in pellet form to alter the melt flow index of the EVA copolymer, which is at least one measure of the rheological performance of the polymer. Further, this example appears to demonstrate a relationship between the radiation dose and effect on melt flow index.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

1-252. (canceled)

253. A controlled release vehicle comprising:
a polymeric matrix having a void space architecture having at least one characteristic selected from the group consisting of a bimodal void diameter distribution, an average void diameter of about 500 microns or less, an average void diameter of about 500 microns or less and a void diameter distribution having a full width at half max of about 50% or less of the average void diameter, an average void distance of about 250 microns or less, an average void distance of about 250 microns or less and a void distance distribution having a full width at half max of about 75% or less of the average void distance, an average pore diameter of about 100 microns or less, an average pore diameter of about 100 microns or less and a pore diameter distribution having a full width at half max of about 50% or less of the average pore diameter, a void space volume of about 95% or less, void density of about 1000 voids per cm³ or greater, and any combination thereof, the polymeric matrix comprising at least one selected from the group consisting of an ethylene copolymer, an ethyl cellulose, a thermoplastic polyurethane, any partially crosslinked polymer thereof, and any combination thereof.

254. The controlled release vehicle of claim 253 further comprising:
at least one agent associated with the polymeric matrix.

255. The controlled release vehicle of claim 253, wherein the polymeric matrix comprises ethylene vinyl acetate copolymer.

256. The controlled release vehicle of claim 253 further comprising:
a polymeric layer disposed on at least a portion of the surface of the polymeric matrix.

257. The controlled release vehicle of claim 253, wherein the polymeric matrix comprises an at least partially crosslinked polymer and is substantially free of chemical crosslinkers.

258. The controlled release vehicle of claim 253, wherein the controlled release vehicle does not comprise a pore forming compound.

259. The controlled release vehicle of claim 253, wherein the substantially open cell void space architecture has a bimodal void diameter distribution.

260. The controlled release vehicle of claim 253, wherein the controlled release vehicle is one selected from the group consisting of a patch, an in vivo implant, a personal care product, a container, a fertilizer, a smoking device, and an insect repellent.

261. A method comprising:
irradiating a plurality of polymer pellets comprising an ethylene copolymer, an ethyl cellulose, a thermoplastic polyurethane, and any combination thereof so as to form a partially crosslinked polymer thereof;
melting the partially crosslinked polymer so as to produce a polymer melt;
extruding the polymer melt through an extruder;
introducing a void forming fluid into the polymer melt while in the extruder; and
forming a controlled release vehicle comprising a polymeric matrix having a void space architecture, the polymeric matrix comprising the partially crosslinked polymer.

262. The method of claim 261, wherein the polymer melt further comprises an agent.

263. The method of claim 261, wherein the polymer pellets comprise ethylene vinyl acetate copolymer.

264. The method of claim 261, wherein the partially crosslinked polymer is at least substantially free of chemical crosslinkers.

265. The method of claim 261, wherein the polymer pellets are exposed to a radiation dose of about 1 mGy to about 50 kGy during irradiating.

266. The method of claim 261 further comprising:
introducing an agent to the polymer melt while in the extruder before introducing the void forming fluid.

267. The method of claim 261, wherein the void space architecture has a bimodal void diameter distribution.

268. The method of claim 261, wherein the void space architecture has an average void diameter of about 500 microns or less.

269. The method of claim 261, wherein the void space architecture has a void space volume of about 95% or less.

270. The method of claim 261, wherein the void space architecture has a void density of about 1000 voids per cm³ or greater.

271. A method comprising:
extruding a polymer melt through an extruder, the polymer melt comprising an ethylene copolymer, an ethyl cellulose, a thermoplastic polyurethane, and any combination thereof;
irradiating the polymer melt while in the extruder so as to form a partially crosslinked polymer thereof;
introducing a void forming fluid into the polymer melt while in the extruder; and
forming a controlled release vehicle comprising a polymeric matrix having a void space architecture, the polymeric matrix comprising the partially crosslinked polymer.

272. The method of claim 271, wherein the partially crosslinked polymer is at least substantially free of chemical crosslinkers.
